(19) United States
(12) Patent Application Publication
(10) Pub. No.: US 2016/0235291 A1
(43) Pub. Date: Aug. 18, 2016

(54) Systems and Methods for Mapping and Evaluating Visual Distortions

(71) Applicant: Dennis Choohan Goh, (US)

(72) Inventors: Dennis Choohan Goh, West Vancouver (CA); Alexander Liam Charles, North Vancouver (CA)

(21) Appl. No.: 14/621,942

(22) Filed: Feb. 13, 2015

(51) Int. Cl. A61B 3/032; A61B 3/00

(52) U.S. Cl. CPC A61B 3/032; A61B 3/0091; A61B 3/0025; A61B 3/0041

(57) ABSTRACT

Systems and methods for mapping and evaluating visual distortions caused by metamorphopsia are provided. A subject with distorted vision is shown initial patterns which may be adjusted to appear non-distorted. A reference pattern within the subject's region of distorted vision is generated from the initial patterns and is used to promote suitable head position and eye fixation. Further patterns within the distorted region are shown to the subject and are adjusted to appear non-distorted. Patterns may be non-adjustable outside of the distorted region. A distortion map is defined based on the adjusted patterns. Additional detail may be added to the distortion map by interpolation. Images may be distorted using the distortion map to appear non-distorted to the subject.

### TECHNICAL FIELD

The present disclosure relates to systems and methods for mapping and evaluating visual distortions, and in particular to systems and methods for mapping and evaluating visual distortions caused by metamorphopsia.

### BACKGROUND

Visual perception is a complex process in which light passes through the cornea, lens, and various humours of the eye before being received by the retina where it is detected by photoreceptors. Nervous signals generated by these photoreceptors are processed by the brain, and more particularly by the visual cortex. A substantial portion of what we perceive as vision is provided by the cognitive processing of the visual cortex. This cognitive processing is not fully developed at birth and must be “learned” during the developmental stages of the visual system (i.e. in the early years of sight). Such cognitive processing includes, for example, mapping stimuli received at particular points of the retina into points of a coherent image. For instance, after cognitive processing, the images of both eyes are perceived as being in registration, inverted images on the retina are perceived as being non-inverted, straight lines are perceived as straight rather than curved (despite the retina itself being curved), and so on.

Metamorphopsia is a condition in which a portion of the retina is displaced relative to its original placement (i.e. its placement during the developmental stages of the visual system). The associated cognitive processing does not generally fully adapt to this displacement, resulting in distortions in the visual perception of images received by the retina. For example, images perceived by a subject with metamorphopsia may be skewed, of a different size, in a different location, or otherwise different than the same images perceived by a subject with a normal retina.

Metamorphopsia has a variety of causes, the most common of which is age-related macular degeneration (AMD). Although there is not currently a cure for AMD, there are a variety of treatments which can slow (or even temporarily reverse) its progression. Such treatments include photodynamic therapy, ocular injections of anti-vascular endothelial growth factor, and nutritional supplements such as lutein, meso-zeaxanthin and zeaxanthin. Accordingly, there is a general desire to detect metamorphopsia in subjects at an early stage. Further, there is a general desire to evaluate the severity of particular cases of metamorphopsia in order to assess the efficacy of a particular treatment.

The quality of life of a subject may be severely and adversely impacted by metamorphopsia (e.g. by a progressively worsening inability to perform daily tasks such as driving, reading, recognizing faces, etc.). Accordingly, it is particularly advantageous in many circumstances to determine the severity of the visual distortions perceived by the subject. The severity of visual distortions may often not be directly determinable from the severity of the physiological displacement of the affected portion of the retina (e.g. as measured via optical coherence tomography), due to the complexities of the cognitive processes discussed above.

Detection of metamorphopsia and the subsequent measurement of the efficacy of treatments are commonly performed by presenting subjects with an Amsler grid, such as the example Amsler grid 100 shown in FIG. 1. Subjects fixate one eye on fixation target 102 and provide anecdotal feedback on, among other things, the size and location of distortions in grid lines 104. This approach can present challenges in ensuring that results are comparable between assessments, since even small variations in the subject's viewing distance, the subject's fixation point, and/or other viewing conditions may significantly impact the perceived size and location of distortions. Further, measurement by conventional Amsler grid may be relatively imprecise.

There have been several recent attempts to collect more precise measurements of visual distortions for the purpose of quantifying, mapping, and (ultimately) correcting the distortions. Some such developments are disclosed, for example, in U.S. Pat. Nos. 5,589,897, 5,892,570, and 8,708,495. Such attempts generally involve providing a pattern which is adjustable by the subject. The subject may then adjust the pattern until it appears “correct” (e.g. until the grid lines of Amsler grid 100 appear straight).

Due to the complexity of the visual system, existing methods may still be prone to inaccuracies, inefficiencies, and/or imprecision in certain circumstances. Accordingly, there is a general desire for systems and methods for mapping and evaluating visual distortions caused by metamorphopsia which ameliorate at least some of the deficiencies discussed above and/or other deficiencies.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

An aspect of the present disclosure provides a method for mapping visual distortions perceived by a subject. The method may be performed by a processor in communication with a display. The method includes displaying, at the display, a fixation target to the subject. The method includes receiving, at the processor, an indication corresponding to an identification of a distortion region by the subject. The distortion region has a location determined relative to the fixation target. The method includes displaying, at the display, an adjustable reference pattern to the subject. The adjustable reference pattern is at least partially within the distortion region and the adjustable reference pattern is adjustable within the distortion region and fixed outside the distortion region. The method includes receiving, at the processor, an indication corresponding to an adjustment by the subject of the adjustable reference pattern within the distortion region. The adjustment is at least partially complementary to visual distortion perceived by the subject in the distortion region. The method includes determining, at the processor, a fixed reference pattern based on the adjustment of the adjustable reference pattern.

The method further includes displaying, at the display, one or more adjustable patterns to the subject. Each of the one or more adjustable patterns is at least partially within the distortion region, and each of the one or more adjustable patterns is adjustable within the distortion region and fixed outside the distortion region. The method further includes receiving, at the processor, one or more indications corresponding to one or more adjustments by the subject of the one or more adjustable patterns within the distortion region. The one or more adjustments are at least partially complementary to visual distortion perceived by the subject in the distortion region. The method further includes determining, at the processor, a distortion map based on the one or more adjustments of the one or more adjustable patterns.

An aspect of the present disclosure provides systems for performing the methods described above. Systems according to particular embodiments may comprise a processor configured to perform one or more of the methods described herein. Non-transitory computer-readable media may be provided with instructions, which (when executed by a suitably configured processor), cause the processor to perform one or more of the methods described herein.

According to another aspect of the invention, the methods described herein are encoded on computer readable media and which contain instructions executable by a processor to cause the processor to perform one or more of the methods described herein.

According to another aspect of the invention, systems are provided wherein processors are configured to perform one or more of the methods described herein.

Further aspects of the invention and features of example embodiments are illustrated in the accompanying drawings and/or described in the following description.

### BRIEF DESCRIPTION OF THE DRAWINGS

The accompanying drawings illustrate non-limiting example embodiments of the invention.

FIG. 1 is an example of a prior art Amsler grid.

FIG. 2A is a schematic cross-sectional illustration of an eye.

FIG. 2B is a graph of visual acuity (on the vertical axis) against retinal eccentricity (on the horizontal axis).

FIG. 2C is a photograph of a portion of the macular region of an example retina.

FIG. 2D is a photograph of a portion of the peripheral region of an example retina.

FIG. 3A is an illustration of an example image received on an example normal retina.

FIG. 3B is an illustration of an example perception of the image of FIG. 3A by an example subject with a normal retina.

FIG. 4A is an illustration of an example image received on an example retina with metamorphopsia.

FIG. 4B is an illustration of an example perception of the image of FIG. 4A by an example subject with a retina with metamorphopsia.

FIG. 5A is an illustration of an example perception of an example image which has been corrected to account for the subject's reported visual distortions, wherein the perception comprises double vision.

FIG. 5B is an illustration of an example perception of an example image which has been corrected to account for the subject's reported visual distortions, wherein the perception comprises an uncorrected portion.

FIG. 5C is an illustration of an example perception of an example image which has been corrected to account for the subject's reported visual distortions, wherein the perception is skewed.

FIG. 6A is an illustration of an example corrected image received on the example retina of FIG. 4A.

FIG. 6B is an illustration of an example perception of the image of FIG. 6A by the example subject of FIG. 4B.

FIG. 7 is a block diagram illustrating an example method for mapping visual distortions.

FIG. 8A is a schematic side elevation view of an example system for mapping visual distortions.

FIG. 8B is a schematic plan view of the system of FIG. 8A.

FIG. 9A is an example calibration display prior to a subject's placement of boundary lines.

FIG. 9B is the example perception of a calibration display of FIG. 9A during the subject's placement of an example first boundary line.

FIG. 9C is the example calibration display of FIG. 9A after the subject's placement of example first and second boundary lines.

FIG. 9D is the example perception of a calibration display of FIG. 9A during the subject's placement of an example third boundary line.

FIG. 9E is the example calibration display of FIG. 9A after the subject's placement of example third and fourth boundary lines.

FIG. 9F is the example perception of a calibration display of FIG. 9A during the subject's placement of an example fifth boundary line.

FIG. 9G is the example calibration display of FIG. 9A after the subject's placement of example fifth and sixth boundary lines.

FIG. 9H is the example perception of a calibration display of FIG. 9A during the subject's placement of an example seventh boundary line.

FIG. 9I is the example calibration display of FIG. 9A after the subject's placement of example seventh and eighth boundary lines.

FIG. 9J is the example calibration display of FIG. 9A after placement of eight example boundary lines and depicting an example region defined therebetween.

FIG. 9K is the example calibration display of FIG. 9A after placement of eight example boundary lines and depicting another example region defined therebetween.

FIG. 9L is the example calibration display of FIG. 9A after a subject's identification of a distortion region according to an alternative embodiment.

FIG. 10A is a grid illustrating various example resolution regions according to an example embodiment.

FIG. 10B depicts various example adjustment rows according to the resolution regions of FIG. 10A.

FIG. 11A is an example adjustment display prior to a subject's adjustment of an example adjustment row.

FIG. 11B is the example adjustment display of FIG. 11A after adjustment of the example adjustment row of FIG. 11A.

FIG. 11C is the example adjustment display of FIG. 11A prior to a subject's adjustment of an example adjustment column.

FIG. 11D is the example adjustment display of FIG. 11A after adjustment of the example adjustment column of FIG. 11C.

FIG. 11E is an example display of an example visual reference based on the adjustment row of FIG. 11B and adjustment column of FIG. 11C.

FIG. 11F is an illustration of an example perception of the display of FIG. 11E by an example subject with a retina with metamorphopsia.

FIG. 12A is an example adjustment display comprising the example visual reference of FIG. 11E prior to a subject's adjustment of a further example adjustment row.

FIG. 12B is an example adjustment display comprising the example visual reference of FIG. 11E after the subject's adjustment of the further example adjustment row of FIG. 12A.

FIG. 13A is an example verification pattern according to an example embodiment.

FIG. 13B is the example verification pattern of FIG. 13A rotated by approximately 45°.

FIG. 14A is an example selection display showing a user selection of a distorted region.

FIG. 14B is an example refinement display showing a constrained region for further adjustment based on the user selection of FIG. 14A.

FIG. 14C is an example perception of the example refinement display of FIG. 14B.

FIG. 15A is an example perception map or distortion map illustrated with distorted gridlines in an example distortion region.

FIG. 15B is an expanded view of a portion of the example map of FIG. 15A illustrating an example method for determining a severity of displacement based on an area displaced.

FIG. 15C is an expanded view of a portion of the example map of FIG. 15A illustrating an example method for determining a severity of displacement based on the displacement of a point from its original (pre-distortion) position.

FIG. 15D is an expanded view of a portion of the example map of FIG. 15A illustrating an example method for determining a severity of displacement based on the local change in displacement of a point.

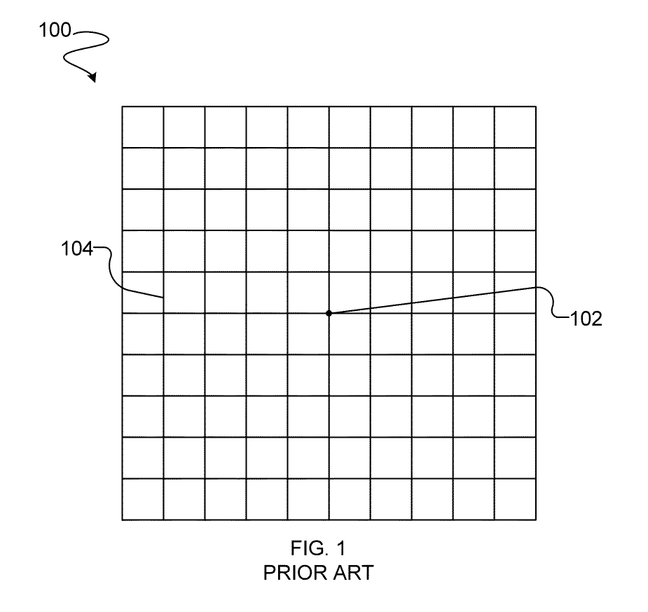

### DETAILED DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive sense.

Methods described herein are implemented by suitably configured computers and/or suitably configured processors. Throughout the disclosure where a processor, computer or computer readable medium is referenced such a reference may include one or more processors, computers or computer readable media in communication with each other through one or more networks or communication mediums. The one or more processors and/or computers may comprise any suitable processing device, such as, for example, application specific circuits, programmable logic controllers, field programmable gate arrays, microcontrollers, microprocessors, computers, virtual machines and/or electronic circuits. The one or more computer readable media may comprise any suitable memory devices, such as, for example, random access memory, flash memory, read only memory, hard disc drives, optical drives and optical drive media, or flash drives. Further, where a communication to a device or a direction of a device is referenced it may be communicated over any suitable electronic communication medium and in any suitable format, such as, for example, wired or wireless mediums, compressed or uncompressed formats, encrypted or unencrypted formats.

One or more processors and/or computers in communication with each other through one or more networks or communication mediums may be collectively or individually referred to herein as a “computer system”. Actions performed by a computer system may be understood to be performed by one or more processors, and/or may be understood to be performed by other components of one or more computers in the computer system (e.g. at an input device such as a keyboard, touch screen, computer mouse, etc.; at an output device such as a display, an audio speaker, a haptic feedback motor, etc.; and/or at other components).

Aspects of the present disclosure provide systems and methods for mapping and evaluating visual distortions caused by metamorphopsia. The methods involve showing a subject initial patterns which may be adjusted to appear non-distorted to the subject. A reference pattern within the subject's region of distorted vision is generated from the initial patterns and is used to promote suitable head position and eye fixation throughout the subsequent steps of the methods. Further patterns within the distorted region are shown to the subject and are adjusted to appear non-distorted to the subject. Patterns may be non-adjustable outside of the distorted region. The adjusted patterns may be used to define a distortion map which allows images to be distorted in a complementary way so that the images appear non-distorted to the subject. The distortion map may undergo interpolation to add detail to the information provided by the adjusted patterns.

Systems according to particular embodiments may comprise a processor configured to perform such methods using a display. Non-transitory computer-readable media may be provided with instructions, which (when executed by a suitably configured processor), cause the processor to perform such methods.

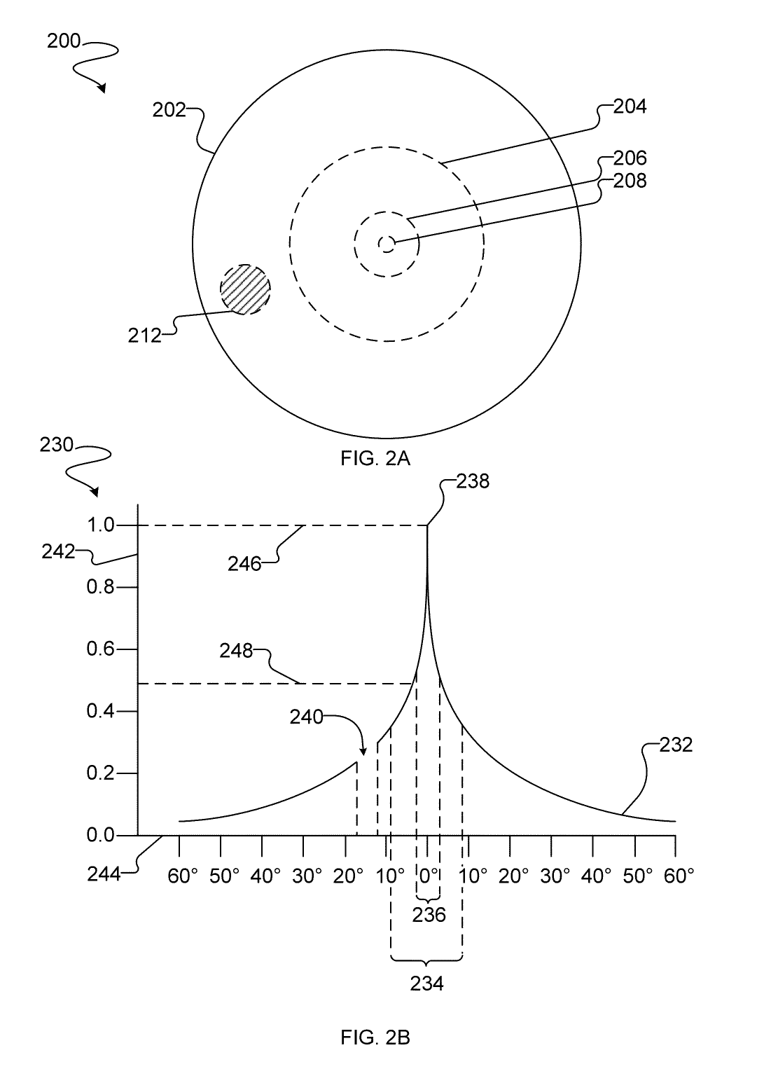

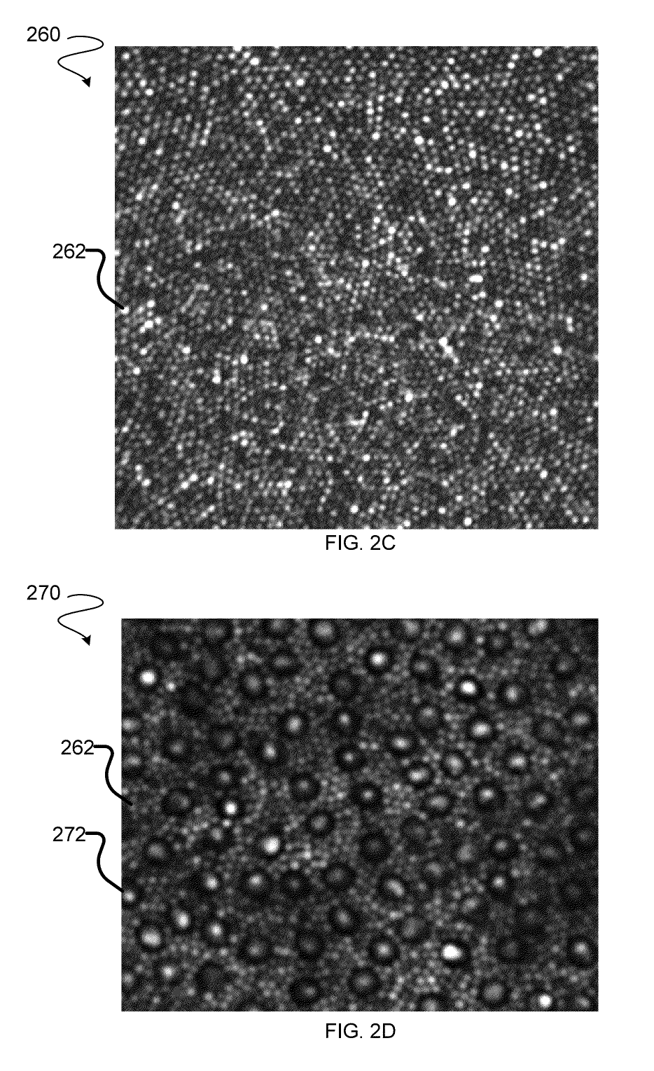

In order to fully appreciate certain aspects of the present disclosure, particular features of the typical human eye should be brought to mind FIG. 2A is a schematic cross-sectional illustration of an example eye 200. The depicted portion of eye 200 is substantially covered by an example retina 202 (and thus substantially opposes the portion of the eye housing the iris, lens, cornea, etc.). Retina 202 comprises several regions, including the macula 204, the fovea 206, and the foveola 208. Retina 202 is partially obstructed by the optic disc 212, which overlays the optic nerve (not shown).

Retina 202 is an arrangement of photoreceptors, which are divided into cones (smaller, densely-packed photoreceptors which provide higher resolution vision as well as colour sensitivity) and rods (larger photoreceptors which provide low light vision and other functions). These photoreceptors are not distributed uniformly or in regular geometric array. In general, cones tend to be more concentrated towards the center of retina 202 (particularly within macula 204), and rods are primarily found further from the center of retina 202. For instance, FIG. 2C is a photograph of a macular region 260 (i.e. located within macula 204 of example retina 202) which primarily comprises cones 262. FIG. 2D is a photograph of a peripheral region 270 (i.e. located outside of macula 204) which comprises a much greater proportion of rods 272.

Due to the greater proportion of cones 262 in macula 204, visual acuity tends to be much greater within macula 204 than outside macula 204. Macula 204 typically has a diameter of approximately 6 mm (i e an angular diameter of approximately 18°). Cones 262 are even more densely packed within fovea 206, which typically has a diameter of approximately 1.5 mm (i e an angular diameter of approximately 5°). Foveola 208 typically comprises only cone photoreceptors, and provides the greatest visual acuity of any region of retina 202 in a typical eye. Foveola 208 typically has a diameter of approximately 0.35 mm (i.e. an angular diameter of approximately 1.2°) Nerve endings are also more densely concentrated in fovea 206 and foveola 208, resulting in much more visual information being transmitted from these areas.

The relative visual acuity of each region of example retina 202 is depicted in chart 230 of FIG. 2B. Axis 242 represents visual acuity, and ranges from 0 (corresponding to no vision) to 1 (corresponding to peak visual acuity, found in the center of foveola 208). Axis 244 represents the angular position of each point in the retina relative to the center of foveola 208. Line 232 depicts the visual acuity of various portions of the retina. For example, point 238 corresponds to the visual acuity of foveola 208 at its center. The portion of line 232 within range 236 corresponds to the visual acuity of fovea 206. The portion of line 232 within range 234 corresponds to the visual acuity of macula 204. Region 240 corresponds to the blind spot coinciding with optical disk 212.

As shown in FIG. 2B, visual acuity drops off sharply outside of foveola 208. Threshold 246 corresponds to maximal visual acuity (e.g. 20/20 vision), which is only achieved in the depicted example at point 238. Threshold 248 corresponds to 50% of the visual acuity at threshold 246, (e.g. 20/40 vision), and is only achieved in the depicted example within approximately 0.75 mm (i e within an angular distance of approximately 2.5°) of the center of foveola 208.

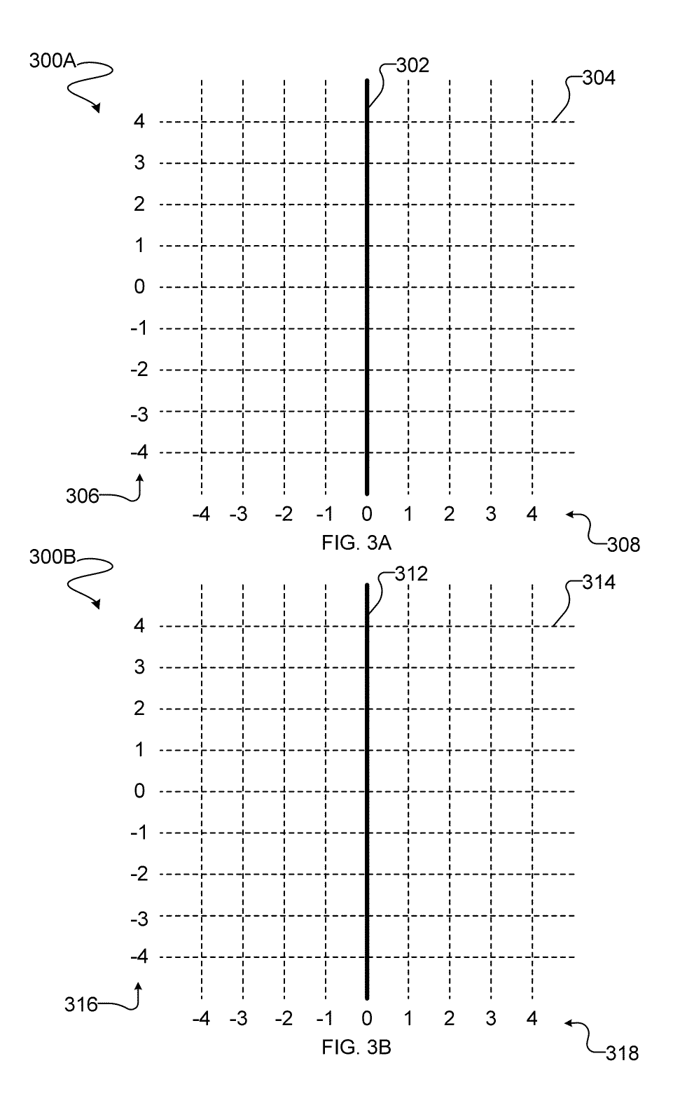

FIG. 3A shows an example image 300A received on an example normal retina. The image comprises, in this example, a straight line 302. Gridlines 304 represent the arrangement of a normal retina without displacement (i.e. without metamorphopsia). Axes 306 and 308 are provided for reference, and correspond to nominal positions on the retina. Line 302 lies substantially straight on the retina (curvature of line 302 due to the shape of curvature of the eye is omitted for the sake of example), which is reflected by the registration of line 302 with gridlines 304. FIG. 3B shows a corresponding perception 300B of image 300A by a subject with a normal retina. Perceived line 312 corresponds to line 302. Perceived line 312 is perceived as straight, as indicated by the registration of line 312 with gridlines 314. Axes 316 and 318 substantially correspond to axes 306 and 308, respectively, although axes 316 and 318 correspond to positions in perceived space (rather than positions on the retina). As would be expected, FIGS. 3A and 3B illustrate an accurate perception of a straight line by a normal eye.

It will be appreciated that, in the above and following examples, straight lines (such as line 302) are used for the simplicity of illustration. Other patterns, including geometric shapes (e.g. squares, rectangles, circles, ellipses, parallelograms, triangles, etc.), irregular shapes (e.g. inkblots, outlines of arbitrary shapes, etc.), photographs, and/or any other suitable visual indicia may be used.

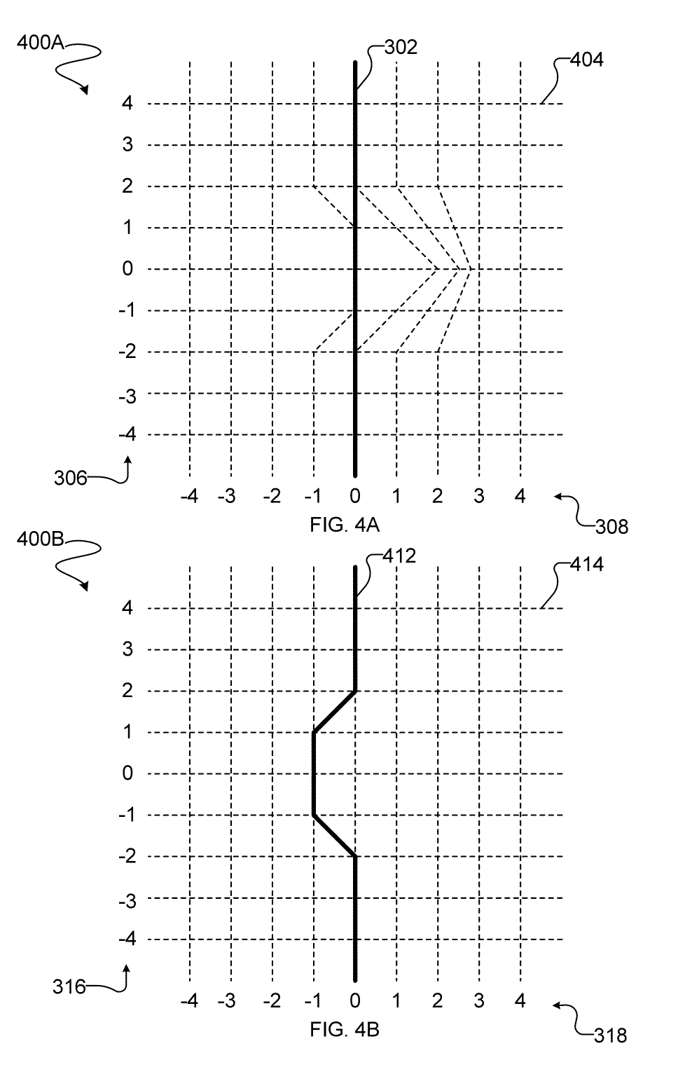

FIG. 4A shows an example image 400A received on an example retina with metamorphopsia. Gridlines 404 are distorted to reflect the displacement of portions of the retina. The same straight line 302 is projected onto the metamorphopsia-affected retina in the same location as in FIG. 3A, although (due to the displacement of a portion of the retina) it is sensed by different photoreceptors. FIG. 4B shows a corresponding perception 400B of image 400A by a subject with the example metamorphopsia-affected retina of FIG. 4A. Due to the displacement of a portion of the retina, perceived line 412 is perceived as being non-straight within the perceived space indicated by gridlines 414. This distortion is due to the physiological displacement of a portion of the retina as well as subsequent cognitive processing which interprets the stimuli received by the retina and transmitted to the brain.

The present disclosure provide systems and methods for mapping the distortions perceived by a subject, thereby generating a distortion map which maps points in an image (such as image 400A) to points in a perceived space (such as in perception 400B). A distortion map, once generated, may be used to “correct” images shown to the subject so that the images are perceived by the subject without distortion. This may be done by distorting images in a manner complementary to the distortions represented by the distortion map. Such corrective distortions (e.g. as represented by a perception map complementary to the distortion map) may be used for diagnostic purposes, to produce corrective optical and/or electronic devices, and/or for other purposes.

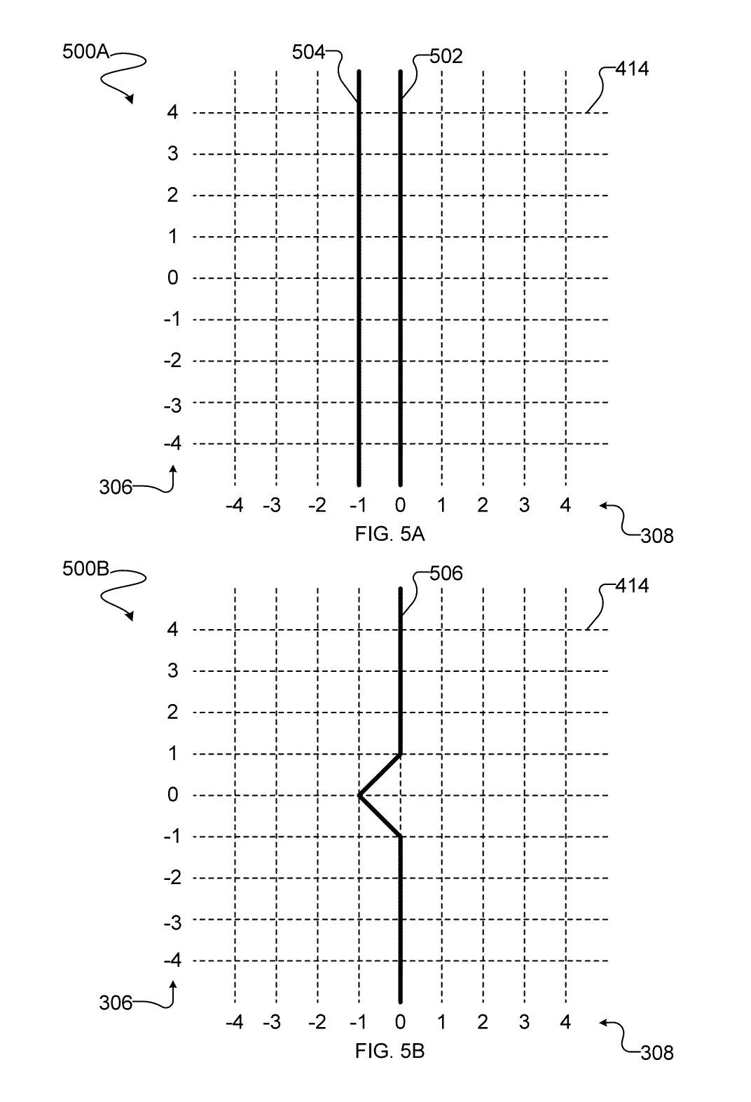

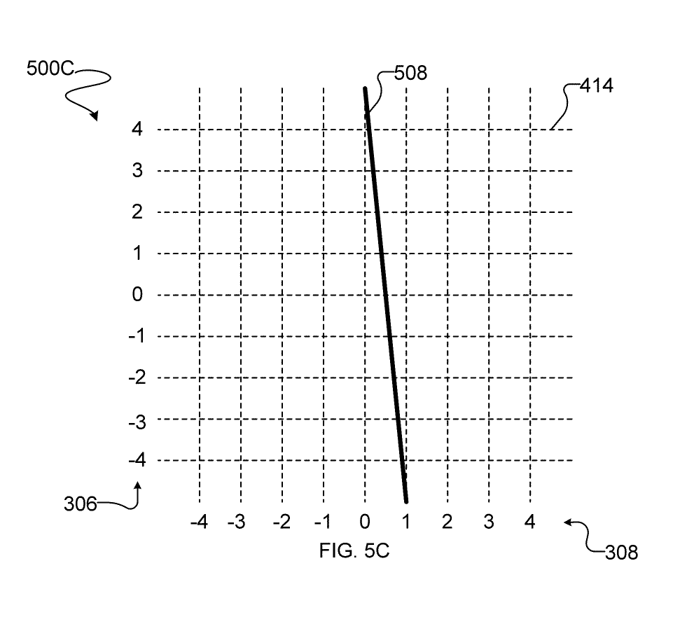

A variety of issues can arise when generating a distortion map and/or when correcting images. In particular, FIGS. 5A, 5B, and 5C show examples of some potential issues.

For example, if the subject's eye is permitted to move during testing, the position of the image on the retina may shift. Such movement is relatively common, since involuntary ocular tremors, ocular drift, and microsaccades may cause the eye to move during the course of testing. In such circumstances, the distortion map may reflect a translational distortion in one eye which is not truly present (or is present to a lesser or greater extent). The same translational distortion may not be determined during testing of the other eye, which may lead to double vision, as shown in perception 500A of FIG. 5A. Rather than perceiving a single straight line 502 corresponding to line 302, the subject may experience double vision, e.g. by perceiving a displaced line 504 in one eye which is out of registration with line 502 in the other eye. Accordingly, in addition to addressing localized distortions (e.g. in addition to straightening a distorted line), it is desirable to ensure that the subject's eye remains fixed on a particular point in the image (e.g. image 400A) during generation of the distortion map so that the corrected image will be properly aligned in both eyes.

FIG. 5B depicts an alternative example perception 500B resulting from a subject's example eye movement. In this case, rather than introducing a translational error, the distortion map may incorrectly reflect a perceived distortion in a portion of the image (e.g. image 400A). For example, a subject may be presented with image 400A (which, as depicted, is perceived as distorted in the range from −2 to 2 along axis 306), and the subject may over-or under-report distortion in a particular location (e.g. in the vicinity of row 0 on axis 306) due to microsaccades, a shift in the subject's fixation point, and/or due to other eye movement resulting in image 400A falling on a different area of the subject's retina. Such eye-movement-induced error may result in an under- or over-correction of the corrected image, resulting in distorted perceptions such as perception 500B.

FIG. 5C depicts an example perception 500C resulting from a subject adjusting portions of a distortion map corresponding to portions of a retina which are not displaced (i.e. portions where there is no true distortion). For example, a subject may adjust distorted portions of an image along the periphery of the image and subsequently adjust remaining portions of the image so as to ensure that the lines remain straight. It is generally more difficult to determine whether lines are parallel, horizontal, vertical or otherwise spatially oriented than it is to determine whether they are straight. This can give rise to a straight perceived line 508 which is out of registration with the perceived space indicated by gridlines 414.

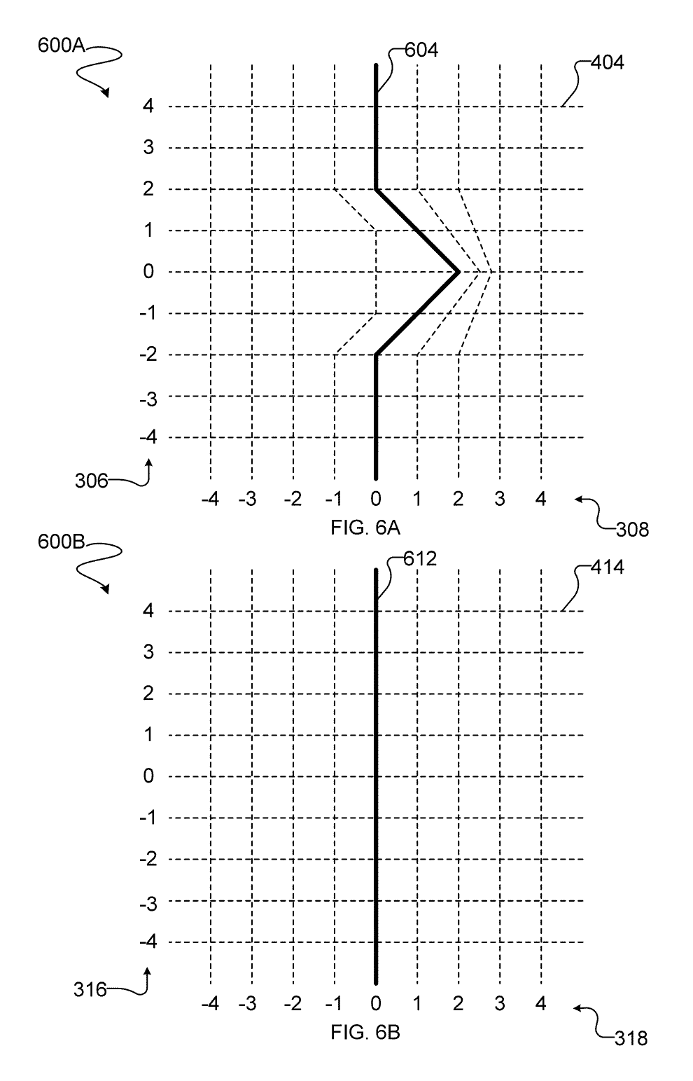

FIG. 6A depicts an example image 600A comprising a corrected line 604. Gridlines 404 correspond to the same metamorphopsia-affected retina as shown in FIG. 4A. In this example, corrected line 604 is perceived as straight line 612, as shown in perception 600B of FIG. 6B. Notably, in this example, corrected line 604 is not merely the geometric inverse of perceived line 412 of FIG. 4B. This helps to demonstrate that the relationship between how a subject may perceive a particular “normal” pattern and its corresponding corrected pattern may be more complex, in some circumstances, than merely finding the inverse of the distorted perception of the pattern. Accordingly, it may be advantageous in some circumstances to generate a perception map from the distortion map, and to generate approximations of images as perceived by the subject based on the perception map.

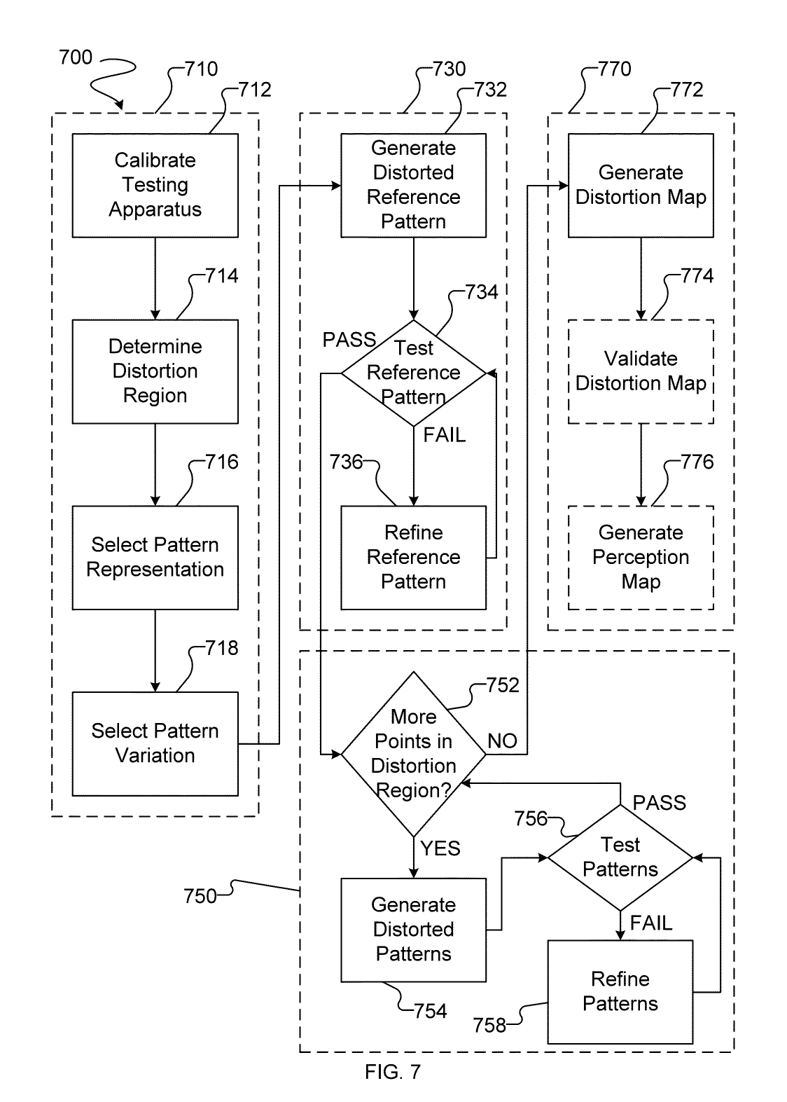

FIG. 7 depicts an example method 700 for generating a perception map. Method 700 may be considered to comprise several sub-methods, namely a calibration method 710, a reference pattern generation method 730, a distortion mapping method 750, and a perception map generation method 770.

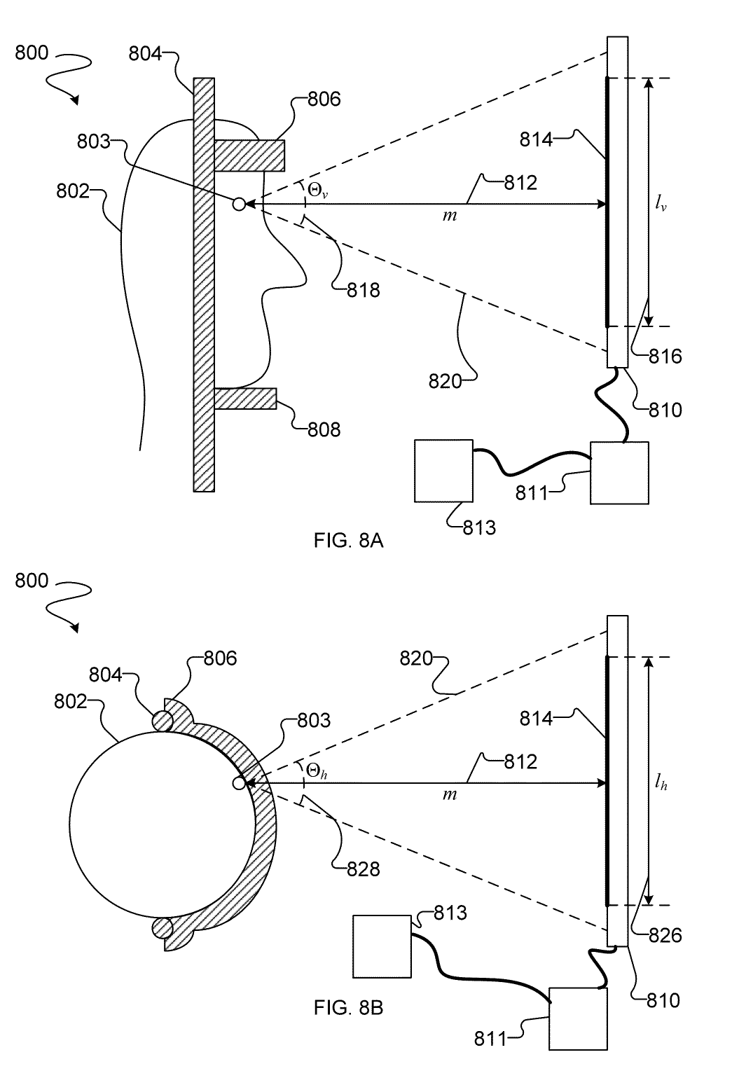

Calibration method 710 comprises block 712 which involves calibrating a system for mapping distortions to present images so that they are received at the subject's retina in a consistent way between mappings. Such a system may, for example, comprise system 800, as shown in FIGS. 8A and 8B. For the purpose of the following disclosure, reference will generally be made to system 800 and its components with the understanding that the methods described herein may be performed by other suitably-configured systems. System 800 comprises an ophthalmic forehead and chin rest, and particularly comprises a support 804 for receiving the head of a subject 802 (subject 802 is not a component of system 800, but is shown for the purpose of better illustrating the functionality of support 804). Support 804 may comprise a chin rest 808 and/or a head rest 806. The head and chin of subject 802 may be secured to chin rest 808 and/or head rest 806 in order to limit head movement (particularly movement towards and/or away from a display 810).

Display 810 is provided facing support 804. Display 810 may be in communication with a computer system 811 (for the purposes of this disclosure, “in communication with” includes being integrated with display 810), which may control display 810 to display graphical indicia such as pattern 814 to subject 802. In some embodiments, display 810 has sufficient resolution to display graphical indicia without significant pixelation, aliasing, and/or other visual artifacts. Display 810 is preferably sufficiently large and positioned sufficiently near to support 804 to accommodate a field of view 820 which includes the subject's distorted areas of vision. In some embodiments, display 810 accommodates a field of view of at least 25° (i.e. having angles 818 and/or 828 of at least 25°) along at least one axis (e.g. along horizontal axis 308). In some embodiments, the center of display 810 is directly in front of the eye 803 of user 802; that is, the surface of display 810 may be perpendicular to the line of sight of user 802.

Further, display 810 is preferably calibrated to provide an accurate image; that is, display 810 is preferably calibrated to display images centered in the screen and geometrically true (i.e. without stretching, compressing, or cropping the image). Display 810 may display images with uniform linear scaling. In some embodiments, display 810 displays images which are adjusted so as to be geometrically true when received at the retina, even though such adjustment may result in the images being deformed at the surface of display 810. For example, a flat display 810 positioned close to a subject may result in an image which is displayed geometrically truly at the surface of display 810 to not be geometrically true at the subject's retina, since the center of display 810 may be closer to the subject's retina than the edges of the display. System 800 may be provided with the distance from the subject's eye to display 800 (e.g. as measured relative to the center of display 810) as well as the size of the display and/or the physical or angular size of the image to be displayed. On the basis of this or other information, system 800 may cause adjusted images 810 to be displayed to the subject which account for angular distortions caused by varying distances between the surface of display 810 and the subject's retina. In some embodiments, if display 810 is known to introduce one or more inaccuracies to displayed images, system 800 may be calibrated to take into account (e.g. counteract) such inaccuracies.

System 800 may be configured to present images to subject 802 with consistent angular sizes. However, displays 810 generally display images based on a linear size (e.g. expressed in pixels, inches, or the like). Therefore, in order to provide consistently-sized images to the retina of subject 802, the physical dimensions of at least a part of system 800 must be known. In some embodiments, distance m indicated by line 812 between eye 803 (or support 804) and display 810 may be predetermined, measured, and/or otherwise determined. For example, distance m may be measured manually and input into computer system 811, and/or computer system 811 may control the position of display 810 and/or support 814 to enforce a particular distance m. In some embodiments, where a range of acceptable distances m are available, display 810 is positioned at the maximum acceptable distance from support 804 (e.g. the largest distance available which provides at least a 25° field of view at display 810).

Once the distance m has been determined, the linear size L of an image displayed on display 810 may be determined according to the following equation:

$$L = 2 m tan \frac{A}{2}$$

where A is the angular size of the image to be displayed on display 810. Although angular measurements are generally presented herein in degrees, they may alternatively or additionally be presented in radians. For example, the above equation is expressed in radians.

Correspondingly, the angular size A of an image having a known linear size L (such as a test image displayed during calibration) may be determined according to the following equation:

$$A = 2 {tan}^{- 1} \frac{L}{2 m}$$

where A, L and m have the meaning discussed above.

In some embodiments, system 811 may display images based on retinal image sizes—that is, the size of an image as received on the retina of subject 802. Based on the angular size A, system 811 may determine the retinal size R of an image according to the following equation:

$$R = L \frac{d}{m}$$

where L and m have the meaning discussed above and d is the distance from the lens to the retina of eye 803 of subject 802. The retinal image size R provides a common basis of measure that may be used across different implementations and different subjects. Retinal image sizes may, in some circumstances, be used in conjunction with distances of retinal disturbances as measured using optical coherence tomography and/or other techniques.

The dimensions l of display 810 and/or a portion of display 810 may be used to drive display 810 to display the image at the appropriate linear size (e.g. if L is intended to be 10 centimeters wide and the width of display 810 is 20 centimeters, then display 810 may be driven to display the image across 50% of the width of display 810). The dimensions l may be predetermined, measured, and/or otherwise determined. For example, a pattern 814 may be displayed on display 810 and measured. For instance, pattern 814 may comprise a straight line stretching diagonally across display 810 (assuming a rectangular display 810) between two predetermined display coordinates. In some embodiments, pattern 814 may be selected to be as large as possible so as to reduce the proportionate magnitude of error in measurement. For instance, pattern 814 may comprise a line stretching from one corner of display 810 to a diagonally-opposing corner. Alternatively, or in addition, dimensions of display 810 and/or a portion thereof, such as vertical dimension 816 (lᵥ) horizontal dimension 826 (lₕ), and/or a diagonal dimension (not shown) may be predetermined or otherwise determined.

Computer system 811 may determine the maximum linear size required to display images in the subsequent tests. If that linear size exceeds the dimensions of display 810, then computer system 810 may reduce the viewing distance m between display 810 and subject 802, display a scaled-down version of one, some, or all images during the test, select a different display 810 of a suitable (larger) size, and/or take other action. Alternatively, or in addition, computer system 811 may present provide direction to an operator to perform one or more of those options. Alternatively, or in addition, computer system 811 may prompt an operator to choose between two or more such options.

At various times this disclosure will refer to subject 802 and/or an operator of system 800 interacting with system 800. It will be understood that subject 802 and/or an operator of system 800 may interact with computer system 811 via input device 813 and/or via any other suitable device (e.g. a computer terminal in communication with system 800, a mobile device, a computer mouse, a joystick, a rotary knob, and/or any other input device known in the art or later discovered). In some embodiments, subject 802 may also be an operator of system 800.

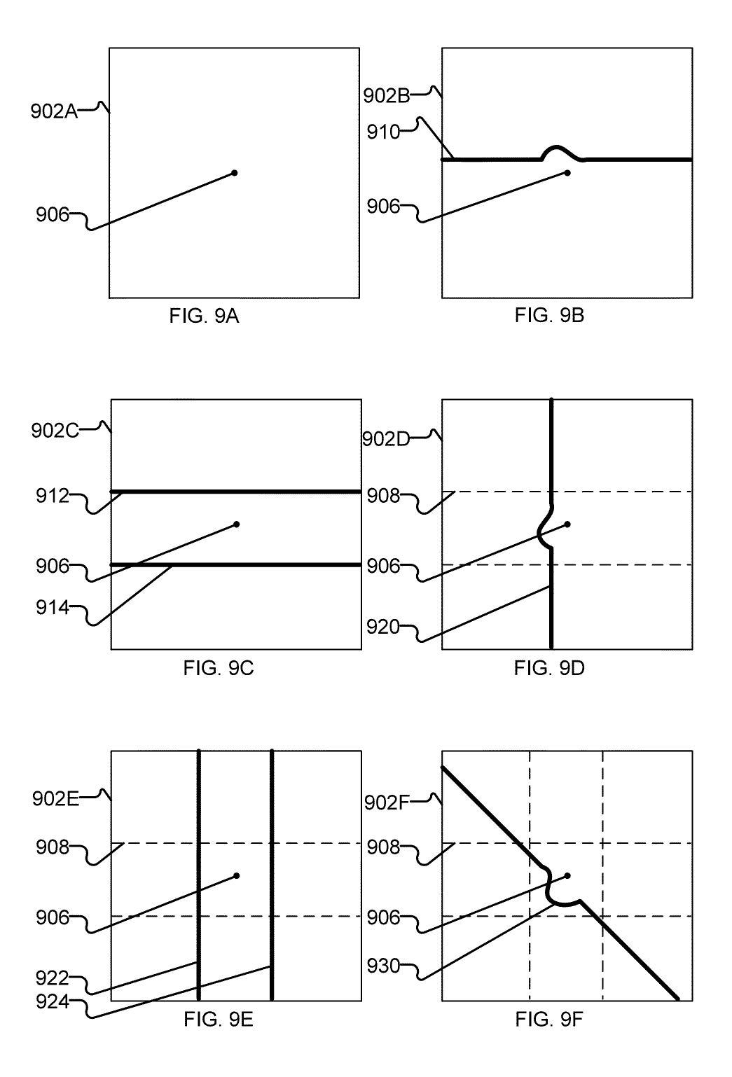

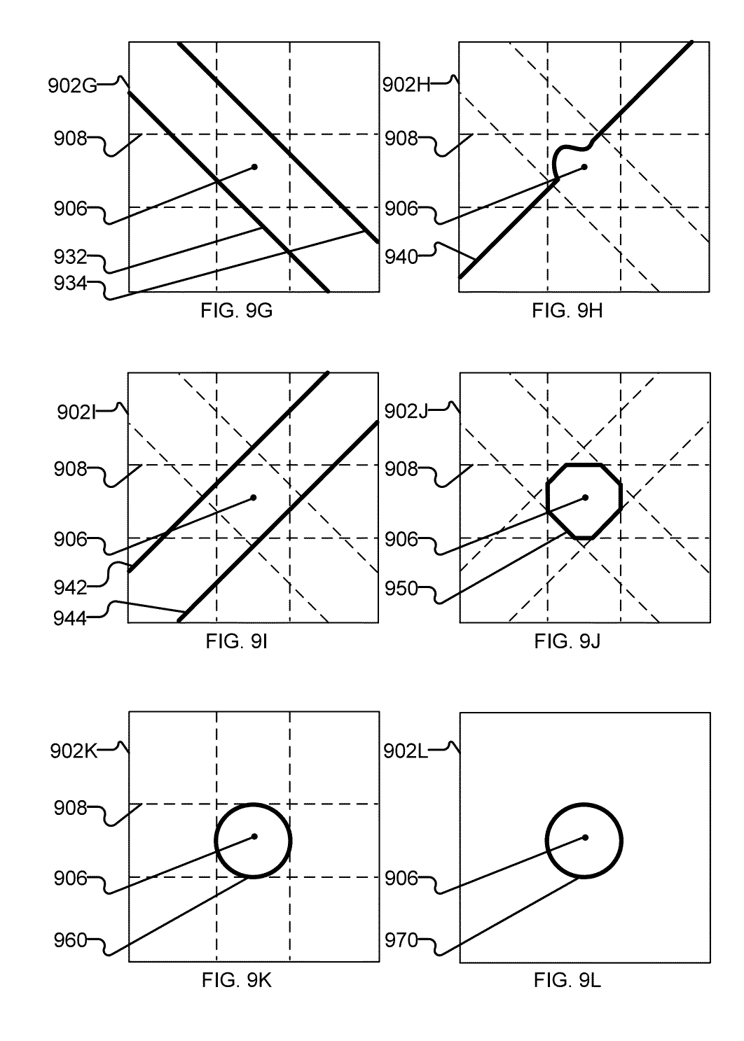

Returning to FIG. 7, once the system (e.g. system 800, as described above) has been calibrated, method 700 may proceed to block 714 which involves determining a distortion region. By determining the region of the field of view 820 of subject 802 which is distorted, method 700 and/or system 800 may prevent subject 802 from adjusting portions of test patterns which lie outside of the distortion region (and thereby avoid introducing certain errors to the distortion map). FIGS. 9A, 9B, 9C, 9D, 9E, 9F, 9G, 9H, 9I, 9J, 9K and 9L illustrate example methods of identifying a distortion region.

FIG. 9A shows an example calibration display 902A (which may be displayed to subject 802 by display 810). Display 902A comprises a fixation target 906 for subject 802 to fixate eye 803 on. Fixation target 906 may comprise a dot, a crosshair (sometimes referred to by the inventor(s) as an “X”), and/or any other suitable shape. Fixation target 906 may have a visual angular size (e.g. an angular diameter) of approximately 0.5°. Fixation targets 906 which are no larger than approximately 0.5° have been found to promote fixation stability in some circumstances.

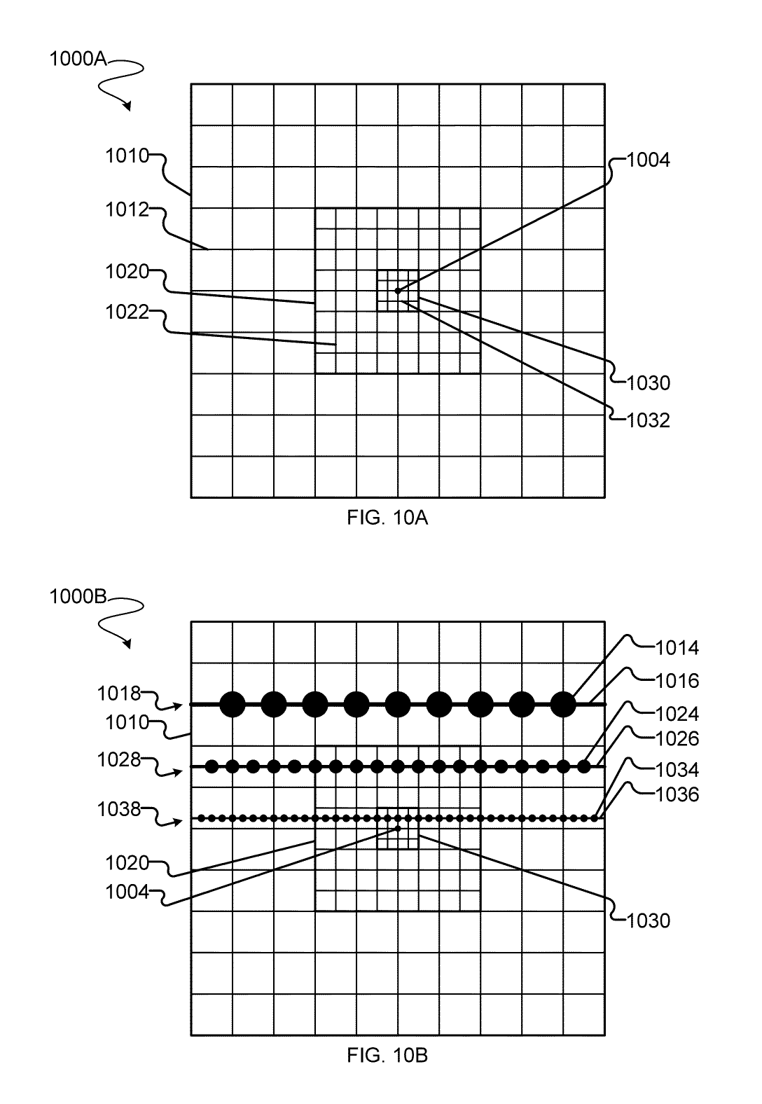

In example display 902A, no boundary lines are shown. In some embodiments, display 902A is not shown to subject 802 (e.g. block 714 may begin with display 902B, discussed below). In some embodiments, display 902A comprises a test pattern (such as an Amsler grid, a multi-resolution grid as shown in FIG. 10A, and/or some other pattern) to assist subject 802 in identifying general areas in field of view 820 where distortions are present. Subsequently, display 902B may be displayed to subject 802.

FIG. 9B shows an example perception of calibration display 902B. Display 902B comprises a boundary line 910. In the depicted example, the perception of boundary line 910 is distorted near to fixation target 906. Boundary line 910 is actually straight, so it can be determined that boundary line 910 passes through a distortion region which happens to be near fixation target 906. Subject 802 may move boundary line 910 until it lies just outside of the distortion region (i.e. until it appears straight). Subject 802 may be constrained in the movement of boundary lines such as boundary line 910 so that boundary lines may only be moved orthogonally to the direction of the line. For example, since boundary line 910 runs horizontally, subject 802 may only be permitted to move boundary line 910 vertically. Subsequently, display 902C may be displayed to subject 802.

FIG. 9C shows an example calibration display 902C. Boundary lines 912 and 914 have been set by subject 802 to lie just outside of the distortion region; boundary lines 912 and 914 lie on opposing sides of that region. For example, boundary line 912 may correspond to boundary line 910 after boundary line 910 has been moved vertically upwards to lie outside of the distortion region. After confirming the location of boundary line 912, subject 802 may similarly set the location of a parallel boundary line 914. Subsequently, display 902D may be displayed to subject 802.

FIG. 9D shows an example perception of a calibration display 902D. Dashed lines 908 correspond to previously-set boundary lines (in this case, boundary lines 912, 914). Dashed lines 908 may be displayed to subject 802 (and may or may not be dashed) or may be hidden from view. A new boundary line 920 is provided. As with boundary line 910, subject 802 may move boundary line 920 until it lies just outside of the distortion region. Boundary line 920 may run in any direction. In some embodiments, and in the depicted example, boundary line 920 runs in a direction orthogonal to previously-set boundary lines 912, 914. Subsequently, display 902E may be displayed to subject 802.

FIG. 9E shows an example calibration display 902E. As with boundary lines 910 and 914, boundary lines 922 and 924 have been set by subject 802 to lie just outside of the distortion region and on opposing sides thereof. Subsequently, display 902F may optionally be displayed to subject 802. Alternatively, the distortion region may be defined based on the area enclosed by lines 908, 922, 924 (see FIG. 9K for an example of this, discussed below).

FIG. 9F shows an example perception of a calibration display 902F. A new boundary line 930 is provided. As with boundary lines 910 and 920, subject 802 may move boundary line 930 until it lies just outside of the distortion region. Boundary line 930 may run in any direction. In some embodiments, and in the depicted example, boundary line 930 runs in a direction diagonal to one or more of the previously-set lines 908. Subsequently, display 902G may be displayed to subject 802, as shown in FIG. 9G. Boundary lines 932 and 934 have been set by subject 802 substantially as described above. Subsequently, display 902H may be displayed to subject 802.

FIG. 9H shows an example perception of a calibration display 902H. A new boundary line 940 is provided. Subject 802 may move boundary line 940 substantially as described above. In some embodiments, and in the depicted example, boundary line 940 runs in a direction diagonal to one or more of the previously-set lines 908 and orthogonal to boundary line 930. Subsequently, display 902I may be displayed to subject 802, as shown in FIG. 9I. Boundary lines 942 and 944 have been set by subject 802 substantially as described above. Subsequently, display 902J may be displayed to subject 802.

FIG. 9J shows an example calibration display 902J. Distortion region 950 is defined between lines 908. In the depicted example, distortion region 950 is determined to be the intersection of the areas between previously-defined pairs of parallel boundary lines 912 and 914, 922 and 924, 932 and 934, and 942 and 944.

FIG. 9K shows an example calibration display 902K. An alternative example distortion region 960 is defined between lines 908 (which correspond to the boundary lines 908, 922, and 924 of FIG. 9E). In the depicted example, distortion region 960 is determined to be an ellipse defined between lines 908. In an alternative embodiment, a distortion region may be determined to be the intersection of the areas between previously-defined pairs of parallel boundary lines 912 and 914 as well as 922 and 924 (which, in this example, would result in a rectangular distortion region), substantially as described above with reference to FIG. 9J.

FIG. 9L shows an example calibration display 902L. An alternative example distortion region 970 is shown. In the depicted example, it is not necessary for boundary lines 908 to have been defined. Distortion region 970 may be defined by a subject 802 selecting a location, size, and/or shape for distortion region 970 (e.g. via input device 813).

It is not necessary that distortion regions 950, 960, 970 exactly correspond to the distortion perceived by subject 802. Indeed, in most circumstances, distortion regions 950, 960, 970 will be slightly larger than the distortion perceived by subject 802. However, it is generally advantageous for distortion region 950, 960, 970 to fully enclose the distortion perceived by subject 802, and to include relatively little of the surrounding non-distorted area.

If subject 802 perceives multiple distortion regions, the above-described process of block 714 may be repeated until all distortion regions of interest have been identified. For the sake of simplicity, the union of all such identified distortion regions will be referred to simply as “the distortion region” in the following disclosure.

Returning to FIG. 7, method 700 (and, more particularly, calibration method 710) may continue to block 716 which involves selecting a representation for patterns to be displayed to subject 802. As is discussed in greater detail below, patterns may be presented to subject 802 during method 700 at various times (including, in some embodiments, during the process of block 716). In some embodiments, these patterns comprise arrays of indicia (such as circles, squares, other geometric shapes, irregular shapes, images, and/or other indicia) which may be connected by a line. One such pattern is shown in FIG. 10B, which provides variously-sized indicia 1014, 1024, and 1034 (in this example, represented as dots) connected by variously-sized lines 1016, 1026, and 1036. Although in some circumstances providing lines is advantageous, in some embodiments indicia are provided without lines, or with other connecting features. In some embodiments, indicia have an angular size in the range of 0.1° to 0.5°, with larger sizes within that range generally being selected for patients with less visual acuity. In some embodiments, connecting lines have a thickness in the range of 0.05° and 0.1°. In some embodiments, connecting lines have a thickness in the range of 5% to 15% the angular size of their corresponding indicia. In some embodiments, connecting lines have a thickness that is not based on the angular size of their corresponding indicia.

The extent and severity of metamorphopsia may vary from subject to subject, as can relative visual acuity. Indicia of different sizes, shapes, and colors may appear to a particular subject to be more or less distorted than other indicia. Different size ratios between indicia and their corresponding lines may also, or alternatively, impact a subject's perception of distortion. Accordingly, subject 802 may be presented with several patterns, simultaneously and/or sequentially. Subject 802 may be prompted to select a pattern from the patterns presented. Computer system 811 may store the selection and subsequently present patterns according to the subject's selection. The patterns may be presented at least partially within the distortion region to assist subject 802 to determine the extent of distortion without having to shift the fixation of eye 803.

In some embodiments, subject 802 may be able to vary aspects of patterns directly; for example, subject 802 may select a line thickness and/or an indicia size (and/or shape, color, and/or other aspects of a pattern) via input device 813. In some embodiments, patterns are predetermined and/or dynamically generated and presented to subject 802 for selection. In some embodiments, subject 802 may provide a selection to an operator of computer system 811 and the operator may input the selection into computer system 811.

As noted above, various patterns are presented to subject 802 during method 700, including during calibration method 710, reference pattern generation method 730, and/or distortion mapping method 750. Patterns may be displayed differently depending on the part of the retina by which they are likely to be perceived. For example, indicia may be presented in smaller sizes in higher-resolution areas of the visual field (the varying acuity of the retina is discussed above, with reference to FIGS. 2A, 2B and 2C). It is generally preferable for the granularity (i.e. resolution) of the pattern in a given location to generally correspond to the acuity of the corresponding portion of the retina (i.e. the portion of the retina on which the image of the pattern falls). The pattern may be presented as a grid divided into a plurality of portions with different resolutions generally corresponding to the acuity of the corresponding portions of the retina. Any number of different portions (and therefore different resolutions) may be provided. In some embodiments, three portions are provided, as discussed further below in reference to FIG. 10A.

FIG. 10A shows an example multi-resolution grid 1000A. Multi-resolution grid 1000A comprises a low-resolution portion 1010 (indicated by sparse grid lines 1012) on the periphery, a medium-resolution portion 1020 (indicated by relatively more dense grid lines 1022), and a high-resolution portion 1030 (indicated by significantly more dense grid lines 1032). In the depicted example, portions 1010, 1020, 1030 are concentric and centered on fixation target 1004. Each portion 1010, 1020, 1030 may comprise a plurality of points (e.g. located at the intersections of grid lines 1012, 1022, and 1032). As will be discussed in greater detail below, subject 802 may adjust patterns based on these points.

Grid lines 1012, 1022, and 1032 are provided to better understand multi-resolution grid 1000A, and are not necessarily displayed to subject 802. Each intersection of grid lines 1012, 1022, and 1032 may correspond to a point in a distortion map. Thus, the distortion map is of higher resolution near to the center of vision of subject 802 (which has greater visual acuity, as illustrated in FIG. 2B), and lower resolution towards the periphery (which has lesser visual acuity, as illustrated in FIG. 2B). In some embodiments, portions 1010, 1020, and/or 1030 are sized to correspond to be angular sizes of macula 204, the fovea 206, and foveola 208, respectively. For example, portion 1030 may have an angular diameter (or, more particularly in the case of the depicted square portion 1030, an angular side-length) of approximately 1°, portion 1020 may have an angular diameter of approximately 6°, and portion 1010 may have an angular diameter in excess of 20°.

In some embodiments, portions 1010, 1020, and/or 1030 may be differently sized and/or differently shaped. For example, portions 1020 and/or 1030 may be circular, elliptical, or otherwise shaped. In general, the preferred size of a particular portion will depend on the acuity of the corresponding part of the retina and on the size of the portion. A portion of the pattern which is much higher-resolution than its corresponding portion of the retina may impose a burden on the subject (since the excess resolution is “wasted” and requires additional and unnecessary attention from the subject), whereas a portion of the patter which is much lower-resolution than its corresponding portion of the retina may result in small distortions (e.g. located entirely between grid lines 1012) remaining undetected and thus uncorrected.

FIG. 10B shows an example multi-resolution grid 1000B with substantially the same features as multi-resolution grid 1000A and patterns overlaid thereon. In particular, a low-resolution pattern 1018 (comprising large indicia 1014 and corresponding line 1016) is overlaid on low-resolution portion 1010, a medium-resolution pattern 1028 (comprising medium indicia 1024 and corresponding line 1026) is at least partially overlaid on medium-resolution portion 1020, and a high-resolution pattern 1038 (comprising small indicia 1034 and corresponding line 1036) is at least partially overlaid on high-resolution portion 1030.

As depicted, each portion 1010, 1020, 1030 has an associated representation for indicia and/or lines therein. In some embodiments, including the depicted embodiment, patterns passing through high-resolution portion 1030 may be represented according to the representation associated with high-resolution portion 1030 even outside of high-resolution portion 1030. Patterns passing through medium-resolution portion 1020 and not high-resolution portion 1030 may be represented according to the representation associated with medium-resolution portion 1020. In some alternative embodiments, a particular pattern may be represented according to multiple representations; for example, a pattern may comprise small indicia 1036 within high-resolution portion 1030 and medium indicia 1026 within medium resolution portion 1020.

In some embodiments, including the depicted embodiment, patterns 1018, 1028, 1038 comprise lines 1016, 1026, 1036, respectively. Lines 1016, 1026, 1036 may assist subject 802 in assessing the straightness of the associated array of indicia 1014, 1044, 1034. In some embodiments, lines 1016, 1026, 1036 are thin relative to their associated indicia 1014, 1024, 1034. For example, each line 1016, 1026, 1036 may comprise a thickness not exceeding 20% of a diameter of the associated indicia 1014, 1024, 1034. Lines 1016, 1026, 1036 may visually connect their associated indicia 1014, 1024, 1034, e.g. by running through the centers of their associated indicia 1014, 1024, 1034. In some embodiments, lines 1016, 1026, 1036 are a different color than their associated indicia 1014, 1024, 1034.

Returning to FIG. 7, block 718 involves selecting a type of temporal variation for the selected patterns. Peripheral indicia (i.e. indicia perceived in the periphery of subject 802's retina) may appear to fade over time due to a phenomenon known as the Troxler effect. It is desirable to prevent or delay the fading of peripheral indicia in order to enable subject 802 to use peripheral indicia as reference points. Causing peripheral indicia to move or otherwise vary in appearance over time may prevent or delay the fading of peripheral indicia. The particular style of variation most suitable to ameliorating the Troxler effect without being unduly distracting may vary from subject to subject. Accordingly, subject 802 may be presented with several styles of variation, simultaneously and/or sequentially. Subject 802 may be prompted to select a style of variation from those presented. Computer system 811 may store the selection and subsequently present patterns with the selected style of variation.

In some embodiments, only indicia which are at least 7° removed from the fixation target (i.e. indicia positioned at at least 7° retinal eccentricity) are made to vary in appearance in the selected manner. In some embodiments, indicia must also, or alternatively, be outside of the displacement region in order to vary in appearance in the selected manner. For example, in some embodiments, only indicia which are at least 7° removed from the fixation target and which are also at least 7° removed from the boundary of the displacement region may be made to vary in appearance in the selected manner.

A variety of variations are possible. A particular variation type which the inventors have found to give good results in some circumstances is movement of indicia (such as indicia 1014 in FIG. 10B) in the direction of their associated line (e.g. along line 1016 in FIG. 10B) once roughly every 5 seconds, alternating between movement in one direction (e.g. left) and the opposing direction (e.g. right). The indicia may move by, for example, half the distance between indicia. Movement may be substantially instantaneous (i.e. indicia may be stationary for 5 seconds, change location between frames and/or between refresh cycles of display 810, and remain stationary for another 5 second before changing location again). Alternatively, or in addition, movement may be animated over a period of time.

Other types of variation are possible. For example, indicia may shift, bounce, stretch, rotate, pulse, grow, shrink, change color, and/or otherwise change their location or appearance. Indicia may move along a path other than (or in addition to) their associated lines, or may move in place (e.g. via rotation).

Calibration method 710 may comprise some or all of the above-identified blocks 712, 714, 716, 718. In some embodiments, certain steps may be omitted or performed in a different order. For example, the block 718 selection of a pattern variation may not be performed, or may be performed before or in parallel with the block 716 selection of a pattern representation. Similarly, the block 716 selection of a pattern presentation may not be performed in some embodiments (e.g. the pattern representation may be predetermined) or may be performed before or in parallel with one or more of block 712 and 714.

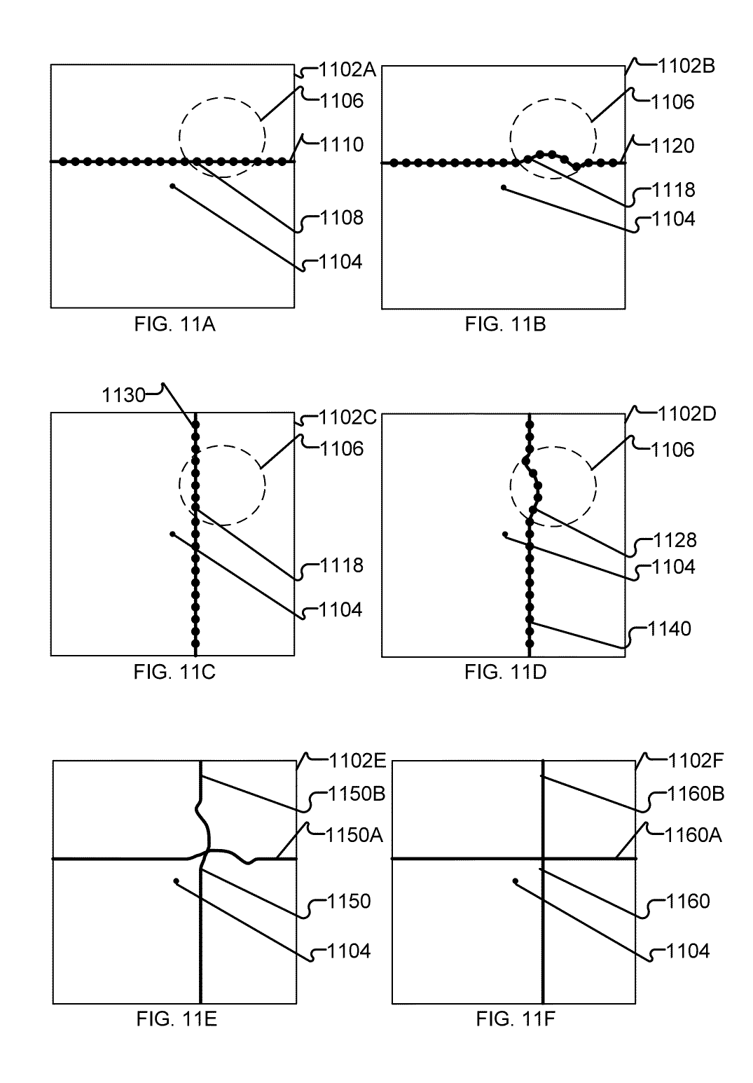

Once calibration method 710 is complete, method 700 may continue to reference pattern generation method 730. Block 732 involves generating an initial reference pattern. The initial reference pattern passes through the distortion region and is adjusted by subject 802 so that the perception by subject 802 of the reference pattern is non-distorted. The initial reference pattern will appear distorted to an observer with a normal retina. The process of block 732 is illustrated in FIGS. 11A, 11B, 11C, 11D, 11D, and 11F (collectively and individually, FIG. 11).

FIG. 11A shows an example adjustment display 1102A having a fixation target 1104 and a distortion region 1106 (distortion region 1106 may be, but is not necessarily, displayed to subject 802). A pattern 1110 passes through distortion region 1106; pattern 1110 comprises indicia and a line substantially as described above.

Pattern 1110 passes through a point 1108. Indicia is displayed at point 1108. In some embodiments, including in the depicted embodiment, point 1108 is the point within distortion region 1106 which is nearest to (is among the points nearest to) fixation target 1104. Pattern 1110 passes through point 1108 in a particular direction (e.g. horizontally). Note that point 1108 may be, but is not necessarily, on the boundary of distortion region 1106. In circumstances where fixation target 1104 is located inside distortion region 1106, point 1108 may be located at fixation target 1104. In such circumstances, fixation target 1104 may be represented by indicia which differ from the indicia of pattern 1110; for example, where pattern 1110 comprises several dots, fixation target 1104 may be represented by a crosshair (and/or an “X”).

Portions of pattern 1110 lying within distortion region 1106 may appear distorted to subject 802. Subject 802 may select particular indicia of pattern 1110 and move the indicia until pattern 1110 appears non-distorted. In the case of a pattern 1110 which comprises a straight line, subject 802 may move the indicia of pattern 1110 until the resulting pattern appear straight. Subject 802 may move the indicia by providing inputs to input device 813, substantially as described above. In some embodiments, computer system 811 allows subject 802 to move indicia within distortion region 1106 but does not permit subject 802 to move indicia outside of distortion region 1106. This restriction may, in some circumstances, prevent subject 802 from introducing certain errors into the distortion map as illustrated, for example, in FIGS. 5A, 5B, and 5C. Subject 802 may be further restricted to moving indicia in a direction orthogonal to pattern 1110 (e.g. vertically).

FIG. 11B shows an example adjustment display 1102B having an adjusted pattern 1120. Adjusted pattern 1120 corresponds to pattern 1110 after subject 802 has moved indicia so as to cause pattern 1120 to be perceived as straight while eye 803 of subject 802 is fixed at fixation target 1104. For example, the indicia formerly at point 1108 is now located at position 1118 (which may or may not be located at a point defined by the multi-resolution grid). As noted above, pattern 1110 (and, therefore, pattern 1120) may be placed near to fixation target 1104; in some circumstances, such a placement may cause subject 802 to be less likely to break fixation with fixation target 1104.

FIG. 11C shows an example adjustment display 1102C having a pattern 1130 orthogonal to pattern 1110 (in this example, vertically). Pattern 1130 passes through point 1118. In some embodiments, any adjustment to point 1108 (resulting in point 1118) may be reflected in pattern 1130; for example, as depicted, point 1118 is slightly displaced, as in FIG. 11B. As described above, subject 802 may adjust indicia in pattern 1130 within distortion region 1106 to make pattern 1130 appear non-distorted (in this example, straight), thereby generating adjusted pattern 1140 shown in FIG. 11D. Adjusted pattern 1140 comprises a point 1128 corresponding to point 1118 after adjustment by subject 802. Subject 802 may be restricted when making adjustments as described above with reference to adjusted pattern 1120.

Once patterns 1120 and 1140 have been determined, an initial reference pattern may be generated. FIG. 11E shows an example display 1102E having an example initial reference pattern 1150 based on adjusted patterns 1120 and 1140. Initial reference pattern 1150 comprises a reference pattern segment 1150A which conforms to the shape of adjusted pattern 1120 (e.g. segment 1150A is coincident with the centers of each of the indicia of adjusted pattern 1120), and a reference pattern segment 1150B which conforms to the shape of adjusted pattern 1140 (e.g. segment 1150B may be coincident with the centers of each of the indicia of adjusted pattern 1140).

Although initial reference pattern 1150 may appear distorted to a person with normal vision, metamorphopsia-affected subject 802 may have perception 1102F as shown in FIG. 11F. That is, subject 802 may perceive initial reference pattern 1150 as a non-distorted perceived pattern 1160. In the depicted example, perceived pattern 1160 comprises segments 1160A and 1160B corresponding to segments 1150A and 1150B, respectively, each of which appears straight to subject 802 when eye 803 of subject 802 is fixed on fixation target 1104, provided that subject 802's head is similarly in registration with display 810.

In some embodiments, segments 1150A and 1150B comprise distorted lines which have different thicknesses than the lines of patterns 1120 and 1140. For example, segments 1150A and 1150B may be approximately 50% thicker than segments 1150A and 1150B. A thickness may be selected based on the subject's preference; for example, a thickness which the subject reports to be relatively less distracting may be selected. By changing the thickness of segments 1150A and 1150B relative to patterns 1120 and 1140, pattern 1150 is made visually distinct from subsequently-displayed patterns (discussed further below).

Although it is possible to provide as many indicia as there are photoreceptors along a given pattern 1120 or 1140, this is rarely practical (since subject 820 would need to adjust potentially tens of millions of indicia). Accordingly, it is generally advantageous to provide a smaller number of indicia in each pattern 1120 and 1140, e.g. in the range from 40 to 60 indicia. A larger number of indicia may be provided when appropriate in the circumstances, such as where greater granularity is required for research purposes, where the distortion region is located around or near to the fovea, where the results of the distortion mapping are intended for use in the calibration of a corrective device with greater granularity, or in other circumstances. For example, the number of indicia may be selected by subject 802 and/or an operator of system 800. The number of indicia may be determined according to the resolution region on which the representation of patterns 1120 and 1140 are based—for example, patterns displayed based on the representation associated with the low-resolution region 1010 may have fewer indicia than those displayed based on the representation associated with the low-resolution regions 1020 and 1030 (as shown, for example, in FIG. 10).

Accordingly, it is generally necessary to derive the locations of portions of segments 1150A and 1150B which are located between the coordinates of the indicia of patterns 1120 and 1140. This derivation may be performed by interpolation. Any suitable interpolation algorithm may be used for this derivation. In some embodiments, Bezier curves and/or splines are used to interpolate the locations of segments 1120 and 1140 between the coordinates provided by the indicia of patterns 1120 and 1140 within distortion region 1106.

Returning to FIG. 7, once initial reference pattern 1150 has been generated, it may be tested at block 734. Such testing may comprise displaying pattern 1150 to subject 802 and confirming whether the pattern appears non-distorted (e.g. as shown in perception 1102F of FIG. 11F) when subject 802 fixes eye 803 on fixation target 1104 and has his/her head in registration with display 810. Computer system 811 may provide a prompt for such confirmation to subject 802 via display 810, via another device, or via an operator of system 811 (e.g. system 811 may prompt the operator to ask subject 811). Subject 802 and/or an operator may provide an input to computer system 811 to indicate whether perceived pattern 1160 appears non-distorted or not.

It may not be necessary, in some circumstances, for subject 802 to perceive pattern 1150 as completely non-distorted. It may be sufficient for perceived pattern 1160 to sufficiently approximate a non-distorted pattern. Accordingly, computer system 811 does not require any particular level of perceived distortion (and, in any event, would likely not be capable of enforcing such a requirement using present technology), but rather receives an indication that the reference pattern either passes or fails to pass the test of block 734.

If computer system 811 receives input which corresponds to a pass (e.g. indicating a substantially non-distorted pattern 1160), method 700 may proceed to block 752 of distortion mapping method 750. Otherwise, if computer system 811 receives input which corresponds to a fail (e.g. indicating a distorted pattern 1160), method 700 may proceed to block 736 of reference pattern generation method 730.

Block 736 involves displaying one or both of adjusted patterns 1120 and 1140 to subject 802 and permitting subject 802 to further adjust patterns 1120 and 1140 substantially as described above. Adjusted patterns 1120 and 1140 may be displayed sequentially and/or simultaneously. If adjusted patterns 1120 and 1140 are displayed simultaneously, subject 802 may be permitted to adjust their point of commonality (i.e. point 1128) in any permitted direction, but adjustments to other points may continue to be restricted as discussed above. Method 700 (and thus method 730) returns to block 734 to repeat the testing of the resulting reference pattern, as described above.

As discussed above, there may be various points within distortion region 1106 (e.g. corresponding to intersections of grid lines 1012, 1024, 1034 on multi-resolution grid 1000A of FIG. 10A), some of which correspond to points on pre-adjustment patterns 1110 and 1130. Method 750 involves generating adjusted patterns for each point in the distortion region (e.g. distortion region 1106) so that each point is associated with each required type of adjusted pattern. For example, computer system 811 may require that each point be associated with a first linear pattern in a given direction and a second linear pattern in an orthogonal direction. For instance, in the depicted examples both horizontal and vertical patterns are used, so method 750 may involve generating horizontal and/or vertical adjusted patterns for each point in region 1106 which is not yet associated with horizontal and/or vertical adjusted patterns. Other types of adjusted patterns may alternatively, or additionally, be used; for example, method 750 may also, or alternatively, require that each point be associated with a diagonal linear pattern, with a non-linear (e.g. circular or elliptical) pattern, or with any other suitable type of pattern.

In the depicted example of FIG. 11, point 1128 is the only point which has corresponding vertical and horizontal adjusted patterns. Other points along adjusted pattern 1120 have only a corresponding horizontal adjusted pattern, so method 750 may involve generating vertical adjusted patterns for each of those points (and similarly generating horizontal adjusted patterns for each of the points other than point 1128 along adjusted pattern 1140). Method 750 may involve generating both vertical and horizontal adjusted patterns for each remaining point in distortion region 1106 which is not located along either of adjusted patterns 1120 or 1140 (or their pre-adjustment counterparts, patterns 1110 and 1130).

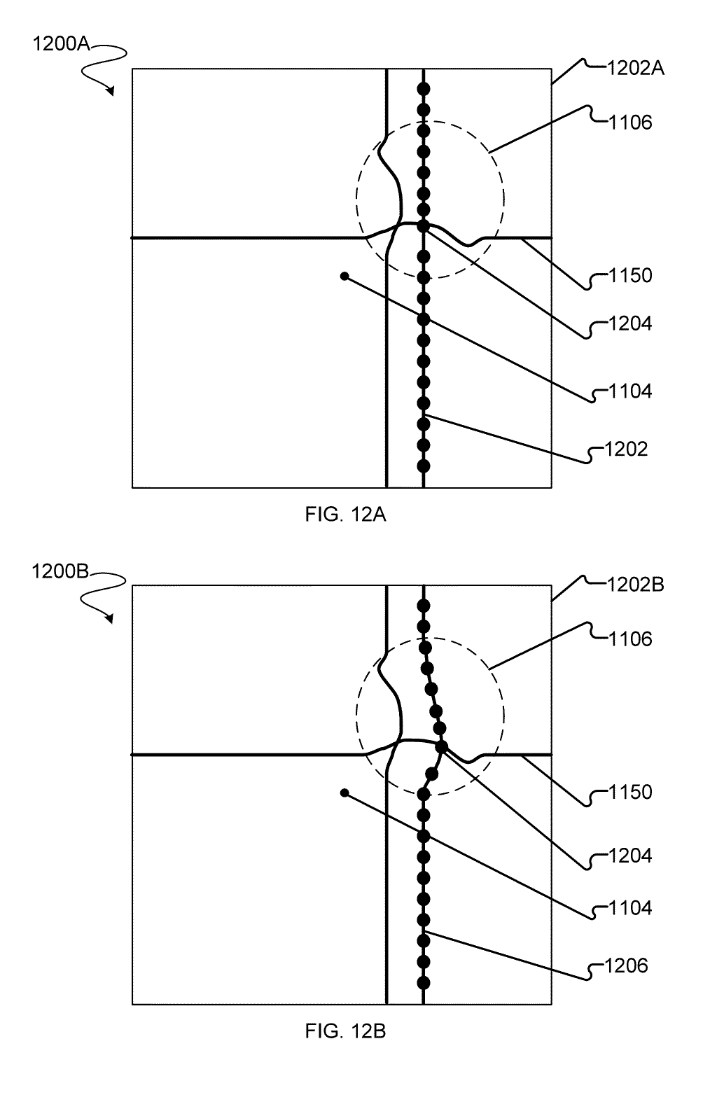

Accordingly, block 752 involves determining whether additional patterns remain to be displayed to (and adjusted by) subject 802 in distortion region 1106. In some embodiments, including the depicted example, this determination comprises determining whether any points within distortion region 1106 do not yet have corresponding adjusted patterns of a required type (e.g. horizontal or vertical). For example, FIG. 12A shows an example point 1204 which is already associated with horizontal adjusted pattern 1140, but is not yet associated with a vertical pattern (which, in this example, is required). FIG. 12A is discussed in greater detail below.

Having identified a point which requires an additional adjusted pattern, method 700 (and therefore method 750) may proceed to block 754. Block 754 involves displaying further patterns to subject 802, similar in some respects to the display of patterns discussed above (e.g. shown in FIG. 11). An example of such a display 1202A is shown in FIG. 12A. Display 1202A has a point 1204 and an additional pattern 1202 which includes point 1204. Note that point 1204 may be positioned according to previous adjustments which have included it, and not necessarily along gridlines 1012, 1022, or 1032. Subject 802 may adjust indicia along pattern 1202 to generate adjusted pattern 1206, as shown in display 1202B of FIG. 12B. This adjustment may be performed similarly to (and subject to the same constraints as) the adjustment of patterns 1110 and 1130, as discussed above.

In the example depicted, point 1204 only requires pattern 1202; in some circumstances, a given point may require multiple additional patterns, each of which may be generated by block 754 substantially as described above (e.g. with reference to block 732). That is, block 754 may generate multiple patterns corresponding to the given point sequentially and/or in parallel. In some alternative embodiments, block 754 may generate only one pattern for a given point, and may generate additional patterns for the given point (if necessary) on subsequent iterations.

In addition to pattern 1202, display 1202A (and/or display 1202B) may also include reference pattern 1140. Reference pattern 1150 may assist subject 802 in ensuring that the head of subject 802 remains in registration with display 810. As described above, if subject 802 fixates eye 803 on fixation point 1104 and keeps his or her head in registration with display 810, reference pattern 1150 should appear substantially non-distorted. Effectively, reference pattern 1150 may provide a mechanism by which subject 802's visual distortions may be used to assist in avoiding common errors in the distortion mapping process, since movements of subject 802's head or eye 802 may result in reference pattern 1150 appearing distorted.

Method 700 (and therefore method 750) may proceed to block 756, which involves testing adjusted pattern 1206 to determine whether adjusted pattern 1206 appears non-distorted (or substantially non-distorted) to subject 802. Such testing may comprise displaying adjusted pattern 1206 to subject 802 and confirming whether the pattern appears non-distorted, as described above with reference to block 736 of method 730. However, block 756 may also involve displaying reference pattern 1150 to subject 802 and confirming that both reference pattern 1150 and adjusted pattern 1206 appear non-distorted. If adjusted pattern 1206 appears non-distorted but reference pattern 1150 appears distorted, it can be concluded that subject 802's eye 803 or head is out of registration with display 810 or fixation target 1104 and that the block 756 test should be repeated (or simply continued, since an input to computer system 811 may not be necessary until a pass or fail has been determined).

If computer system 811 receives input which corresponds to a pass (e.g. indicating a substantially non-distorted pattern 1206 and reference pattern 1150), method 700 may proceed to block 752 of distortion mapping method 750. Otherwise, if computer system 811 receives input which corresponds to a fail (e.g. indicating a distorted pattern 1206 while reference pattern 1150 appears non-distorted), method 700 may proceed to block 758.

Block 758 involves displaying one or more of the adjusted patterns generated by block 754 (e.g. adjusted pattern 1206) to subject 802 and permitting subject 802 to further adjust the pattern(s) substantially as described above with reference to block 736. As with block 754, reference pattern 1150 may be displayed during the further adjustment of the pattern(s). Method 700 (and thus method 750) returns to block 756 to repeat the testing of the resulting adjusted patterns, as described above.

Once all of the points in distortion region 1106 have corresponding adjusted patterns of the required types, method 700 may proceed to perception map generation method 770, and in particular to block 772. Block 772 involves generating a distortion map from the adjusted patterns generated by methods 730 and/or 750 (such as reference pattern 1150 and adjusted pattern 1206). The union of the adjusted patterns may provide a grid with distorted lines which, when viewed by subject 810 while fixating eye 803 on fixation target 1104 and with the head of subject 802 in registration with display 810, appears substantially non-distorted. These grid lines provide a map from “normal” visual space to “corrected” visual space. For example, if point 1128 corresponds to point 1108 prior to subject 802's adjustments, then the coordinates of point 1108 may be mapped to the coordinates of point 1128.

For example, if point 1108 has coordinates of (100, 100) relative to fixation target 1104 (in (X,Y) coordinate format), and if point 1128 has coordinates of (110, 105) in the same format, then an image may be mapped from “normal” visual space to “corrected” visual space by mapping elements (e.g. pixels) of an image located at (100, 100) to the new location (110, 105). If all elements along pre-adjustment patterns 1110, 1130, 1202, etc. (including elements along the lines thereof) are mapped to the corresponding coordinates along adjusted patterns 1120, 1140, 1206, etc. (based on the above-described interpolated lines), then the “corrected” image may appear non-distorted to subject 802 along at least the perceived areas corresponding to adjusted patterns 1120, 1140, 1206, etc. (although those areas may still appear distorted if neighbouring areas are distorted, due to the complexities of cognitive processing).

Accordingly, a partial distortion map may be generated by mapping coordinates of all pre-adjustment patterns (such as patterns 1110, 1130, 1202, etc.) to the corresponding coordinates along adjusted patterns 1120, 1140, 1206, etc. However, in many circumstances it is desirable to generate a more detailed distortion map which also maps locations not on patterns 1110, 1130, 1202, etc. to locations not on adjusted patterns 1120, 1140, 1206, etc.

Method 770 (and in particular block 772) may involve mapping such coordinates by interpolation. For example, coordinates within distortion grid 1106 may be mapped from “normal” visual space to “corrected” visual space by applying bicubic interpolation based on the partial distortion map. Thus, each coordinate in “normal” visual space not already mapped to a coordinate in “corrected” visual space by the partial distortion map may be mapped to such a coordinate based on the mappings of the surrounding coordinates (in “normal” visual space). The mapped coordinates may be included in the generated distortion map. Other types of interpolation may be used. Preferably, two-dimensional interpolation methods may be used, such as bilinear interpolation, bicubic interpolation, Bèzier surfaces, and/or other interpolation methods.

Once a distortion map has been generated, method 700 (and therefore method 770) may optionally proceed to block 774, which involves validating the distortion map generated at block 772 to confirm that the generated distortion map is sufficiently accurate; that is, to confirm that images mapped from “normal” space to “corrected” space by applying the distortion map are perceived by subject 802 as substantially non-distorted. This validation may comprise displaying a validation pattern.

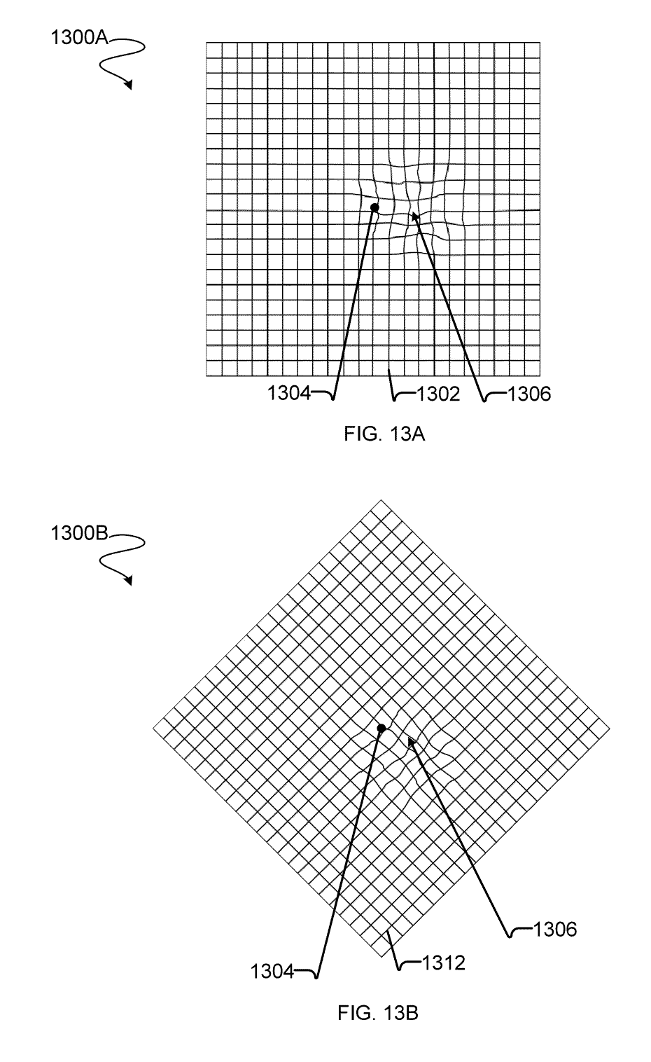

FIG. 13A shows an example validation pattern 1300A having a fixation target 1304 and grid lines 1302. Grid lines 1302 do not necessarily correspond to grid lines 1012, 1022, 1032 of grid 1000A. An area 1306 has been distorted according to the distortion map so that all grid lines 1302 should be perceived as straight by subject 802. For instance, validation pattern 1300A may comprise a conventional Amsler grid which has been distorted according to the distortion map. Alternatively, or in addition, other validation patterns such as other patterns, photographs, or other images may be displayed to subject 802.

Block 774 may comprise varying the displayed validation patterns. For example, block 774 may comprise rotating the validation pattern, updating the distorted area 1306 to reflect any movement or other change in the underlying pattern. For example, the Amsler grid of FIG. 13A may be rotated about fixation point 1304 through 90°. FIG. 13B shows an example validation pattern 1300B which corresponds to the Amsler grid of FIG. 13A rotated 45°, with the distortion map applied in area 1306 to distort the grid lines of the rotated grid according to the distortion map (which is not rotated with the validation pattern). Other validation patterns, such as those with a different degree of rotational symmetry, may be rotated more or less—for instance, a validation pattern without rotational symmetry may be rotated through a full 360°. Reference pattern 1150 may be displayed during the display of validation patterns 1300A, 1300B, as described above.

As with blocks 734 and 736, computer system 811 may receive an input indicating whether the validation passes or fails—e.g. if subject 802 perceives a distortion during the rotation of validation patterns 1300A, 1300B, then computer system 811 may be provided with an input which indicates failure. In response to receiving an input indicating that the validation passes, method 700 (and therefore method 770) may proceed to block 776. Otherwise, if the computer system 811 receives an input indicating that the validation fails, block 774 may involve further refinement of the distortion map, as described below.

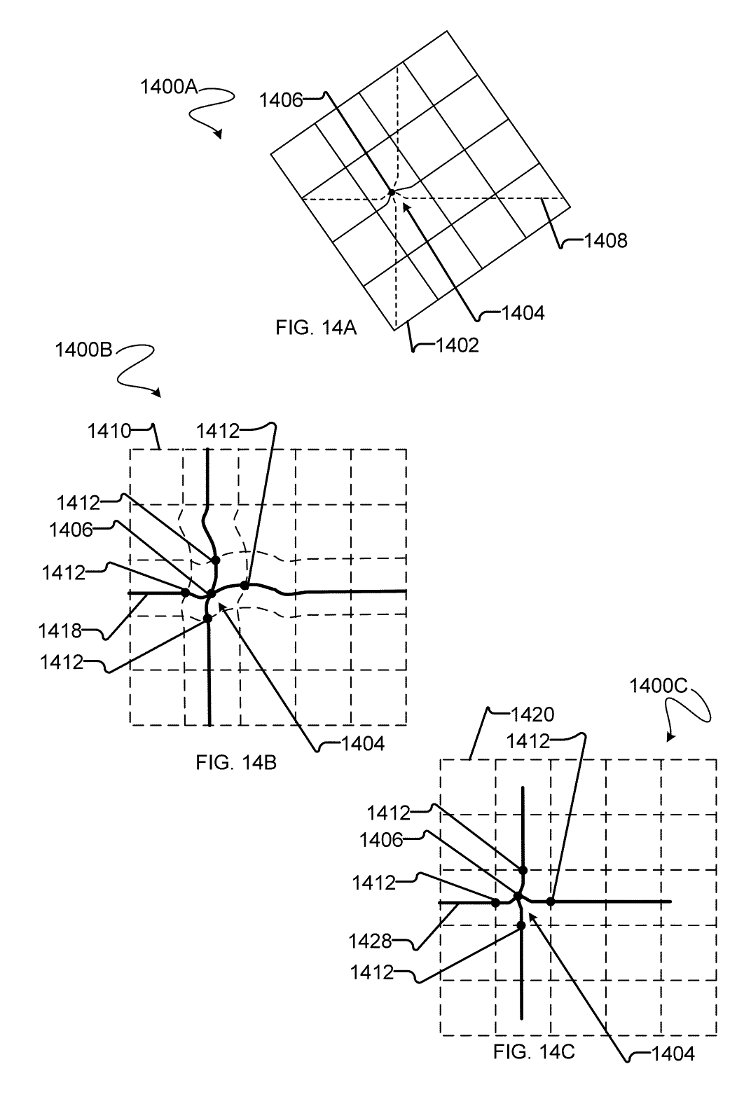

If a portion of a validation pattern is perceived by subject 802 to be distorted, a failure indication may be provided to computer system 811. Subject 802 may then select a region of the validation pattern which appears distorted and make further adjustments. For example, FIG. 14A shows an example perception 1400A of a selection display showing a perceived portion 1402 of validation pattern 1300B. Portion 1402 generally corresponds to area 1306. Area 1404 has distorted according to the distortion map (and so should, ideally, appear non-distorted), but subject 802 nevertheless perceives area 1404 as distorted. Subject 802 may select a point where a distortion is perceived. In this example, subject 802 has selected (e.g. via input device 813) point 1406 in area 1404. Reference pattern 1150 (not shown) and/or fixation target 1106 (not shown) may continue to be displayed as described above.

In some embodiments, a guide pattern 1408 may be displayed to subject 802 to assist subject 802 in selecting a point. For example, guide pattern 1408 may comprise a crosshair which has been distorted according to the distortion map. Guide pattern 1408 may be displayed instead of, or in addition to, a user interface element such as a mouse cursor. Subject 802 may move the intersection of the crosshair to point 1406 (or to any other point in the distortion region) and select point 1406 using input device 813 or any other means. Guide pattern 1408 may be easier for subject 802 to identify than non-distorted user interface elements in areas where the distortion map is accurate. In some embodiments and in some circumstances, guide pattern 1408 may assist subject 802 in identifying areas where the distortion map is inaccurate by essentially providing a dynamic reference pattern in areas of interest. For example, as shown in FIG. 14A, guide pattern 1408 is perceived as distorted in area 1404.

In some embodiments, guide pattern 1408 comprises lines which are thinner than the lines of patterns 1110 or 1130. For example, guide pattern 1408 may comprise lines which are less than half as thick as the lines of patterns 1110 or 1130. Guide pattern 1408 may also, or alternatively, be in a different colour than the lines of patterns 1110 or 1130 and/or validation pattern 1402.

Once point 1406 has been selected, a refinement display 1400B may be displayed to subject 802, as shown in FIG. 14B. Refinement display 1400B may include point 1406 and/or interpolated pattern 1418. Interpolated pattern 1418 may be substantially similar to guide pattern 1408. For example, interpolated pattern 1418 may comprise a crosshair which has been distorted according to the distortion map. The distortion map may comprise grid lines 1410, which are not necessarily shown to subject 802. Grid lines 1410 may comprise adjusted patterns 1120, 1140, 1206, etc. Anchor points 1412 may be placed at the points where interpolated pattern 1418 intersects with grid lines 1410. Interpolated pattern 1418 may be derived from the distortion map, the interpolation of which is described above. In some embodiments, multiple interpolated patterns 1418 may be displayed to the subject; each interpolated pattern 1418 may be generated using a different interpolation algorithm. For example, one interpolated pattern 1418 may be generated using bilinear interpolation, another using bicubic interpolation, another using Bezier surfaces, and so on. The subject may select any one of the presented interpolated patterns 1418 for use by system 800; the selected interpolated pattern 1418 may be further refined as discussed below.

FIG. 14C shows an example perception 1400C of display 1400B by subject 802. Grid lines 1420 correspond to distorted grid lines 1410. Although grid lines 1420 are not necessarily visible to subject 802, if they were visible they would likely be perceived to be straight (since they correspond to previously-tested adjusted patterns 1120, 1140, 1206, etc.). Perceived pattern 1428 is subject 802's perception of interpolated pattern 1418. Subject 802 perceives a distortion in perceived pattern 1428, as is clearly visible in FIG. 14C.

Subject 802 may be permitted to adjust the position of point 1406 so that interpolated pattern 1418 appears substantially non-distorted at least in the area bounded by anchor points 1412. Adjustment of the position of point 1406 may be performed substantially as described above with reference to blocks 736, 754 and points 1128 and 1204. Further points along interpolated pattern 1418 between point 1406 and anchor points 1412 may be defined and may be adjusted by subject 802. In response to an adjustment of the position of point 1406 (and/or other points) by subject 802, computer system 811 may reinterpolate portions of pattern 1418. In some embodiments, only the portions of pattern 1418 which are bounded by anchor points 1412 are reinterpolated (i.e. the effects of adjustments may be confined to the cell defined by grid lines 1410 in which the adjustment occurs).

The resulting adjusted and/or reinterpolated pattern may, optionally, be tested substantially as described with reference to block 756 and, if computer system 811 receives an input indicating that the test is passed, the reinterpolated pattern may be incorporated into the distortion map as if it were an adjusted pattern 1120, 1140, 1206, etc. Coordinates surrounding the reinterpolated and/or adjusted pattern may be used as a basis for further reinterpolation of the distortion map. For example, coordinates not on the reinterpolated pattern but within the area bounded by grid lines 1410 on which anchor points 1412 are defined may have their mappings reinterpolated.

Validation pattern 1402 need not necessarily displayed to subject 802 during refinement of interpolated pattern 1418. Reference pattern 1150 (not shown) and/or fixation target 1106 (not shown) may continue to be displayed as described above.

Once generated (and optionally validated and/or refined), a distortion map may be used for various purposes. For example, a distortion map may be used to generate corrective optics, such as shaped lenses, GRIN lenses, and the like. As another example, distortion maps may be used to generate corrective displays, such as HUDs with eye trackers, adaptive vision goggles, video displays, and the like. As a further example, distortion maps may be used to generate quantitative measures of the physical displacement of the retina of a subject 802, which may assist in researching and/or treating metamorphopsia.

In some embodiments, after generating a distortion map at block 772 (and/or, optionally, after validating the distortion map at block 774), method 700 proceeds to block 776, which involves generating a perception map. Unlike a distortion map, which allows for a mapping of “normal” images to “corrected” images which are perceived as substantially non-distorted by subject 802, a perception map enables a person with normal vision to perceive distortions substantially as they are perceived by metamorphopsia-affected subject 802.

As illustrated, for example, by FIGS. 4B and 6A, a perception map is not necessarily the same as the geometric inverse of a distortion map. For instance, at the 0 index along axis 306, perceived line 412 is offset from the position of line 302 by −1 unit, but the geometric inverse of line 604 at the same location would result in an offset of −2 units.

The block 776 generation of a perception map may involve finding a functional inverse of the distortion map. That is, given a distortion map D: N→C, where N is the “normal” visual space and C is the “corrected” visual space which complements the distortion of subject 802, and given that D (I) for an arbitrary image I is perceived by subject 802 as substantially non-distorted, then the perception map P: C→N can be defined as P(I)=D⁻(I), so that P(D(I))=I. In other words, given a coordinate pair (x,y) such that D((x,y))=(x′,y′), the perception map can be defined as P((x′,y′))=(x,y).

In some embodiments, computer system 811 stores the distortion map at least partially as a lookup table, and generates the perception map by performing a reverse lookup and/or by reversing the lookup table.

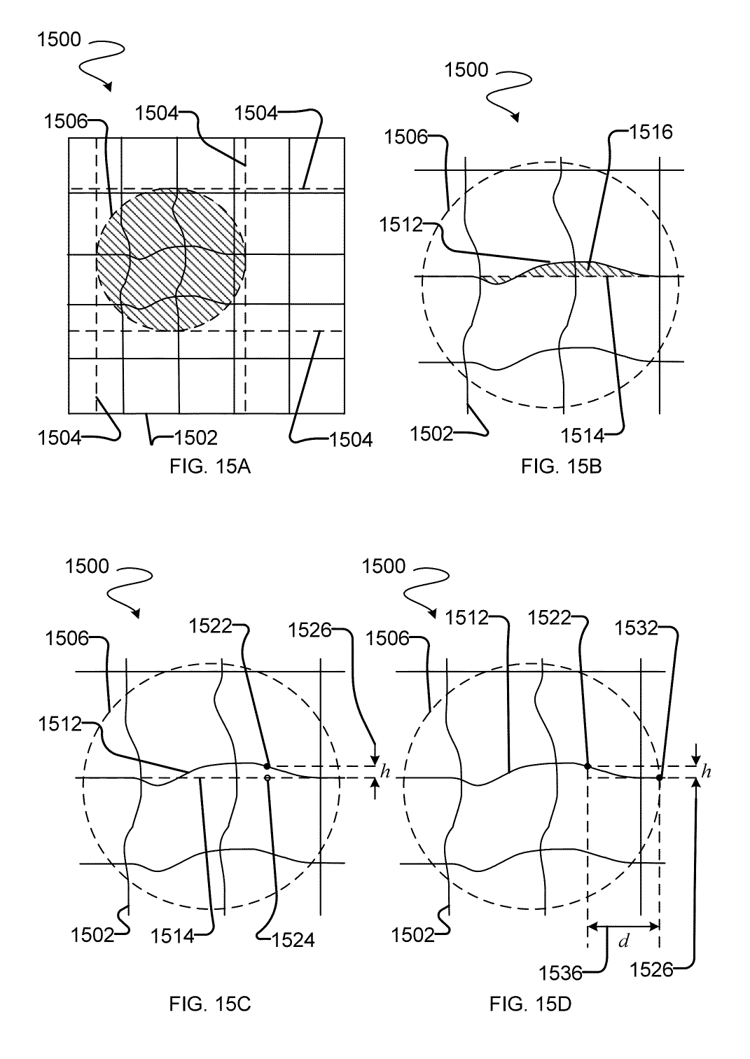

Once generated, a perception map may be used for various purposes. For example, a perception map may be used as a tool by those who work with metamorphopsia-affected individuals as part of a treatment, rehabilitation, and/or the like. As another example, a perception map may be used to determine a quantitative measure of the visual distortions perceived by a subject 802, which may be relevant to assessing the overall quality of vision (and/or quality of life) of a subject 802. Quantitative measures may also, or alternatively, be generated from distortion maps; although a perception map may better approximate the subjective distortions perceived by a subject 802, it may be convenient or desirable in some circumstances to generate quantitative measures from a distortion map. System 811 may provide one or more quantitative measures of the visual distortions perceived by subject 802. Certain methods of determining quantitative measures are discussed below, with example methods illustrated in FIGS. 15A, 15B, 15C, and 15D.

In some embodiments, system 811 may determine a quantitative measure based on an area of the distortion region identified by subject 802 (e.g. distortion region 950, 960, and/or 970 of FIGS. 9J, 9K, and 9L). FIG. 15A shows an example map 1500 (which, as discussed above, may be a perception map or a distortion map). Grid lines 1502 are shown for the sake of clarity, although, as discussed above, the distortion and perception maps may be interpolated to provide a mapping which includes points not located on grid lines 1502. Subject 802 has identified boundary lines 1504 which contain the distortion region, as described above. A quantitative measure Q may be determined based on the area of the distortion region, e.g. so that distortion regions with larger areas may correspond to quantitative measures reflecting more severe distortions.

Due to the physiological nature of metamorphopsia, the actual distortion perceived by subject 802 is unlikely to be bounded by straight lines. Most commonly (although not universally), the actual distortion perceived by subject 802 is likely to be better-approximated by an ellipse than by a linearly bounded distortion region. System 811 may determine an ellipse 1506 which approximates the distortion region, and may determine a quantitative measure based on the area of ellipse 1506. In some embodiments, ellipse 1506 may be bounded by the distortion region identified by subject 802. For example, a quantitative measure Q may be determined according to the formula:

Q=ƒ(π×a×b)

where a is the radius of the major axis of the ellipse, b is the radius of the minor axis of the ellipse, and ƒ is a function which takes the area of the ellipse as an input. ƒ may be the identity function.

In some embodiments, system 811 may determine the quantitative measure based on the severity of displacement of all or part of the distorted region. FIGS. 15B, 15C and 15D show an expanded view of a portion of example map 1500, namely the portion in the vicinity of the distortion region. In some embodiments, system 811 selects a line 1514 as displayed at display 810 (i.e. prior to being perceived and distorted) passing through the distortion region. Line 1512 is the perception of line 1514 by subject 802; as shown, line 1512 is distorted so that various points therein are displaced relative to line 1514. System 811 may determine a quantitative measure based on the severity of the displacement caused by this distortion.

In some embodiments, the severity of displacement may be determined based on an area displaced. FIG. 15B illustrates an example method for determining a severity of displacement based on an area displaced. System 811 may determine the severity (and thus the quantitative measure Q) based on the area 1516 between original line 1514 and distorted line 1512. Such an area may be approximated, for example, by summing the displacement of indicia along line 1512 relative to line 1514, by integrating along the curve of line 1512 (e.g. using numerical integration methods, such as Simpson's rule, and/or symbolic integration methods) to determine the approximate area between lines 1512 and 1514, and/or by other methods. For example, a quantitative measure Q may be determined according to the formula:

Q=ƒ(∫ₐ^{b}|g(x)|dx)

where a and b are points which bound the distorted portion of the linear cross-section (e.g. a and b may be on opposing sides of the boundary of the distorted region, on opposing sides of display 1202B, or elsewhere), g(x) is the displacement of point x on the linear cross-section, and ƒ is a function which takes the area of displacement as an input. ƒ may be the identity function.

As another example, system 811 may determine the severity of the displacement based on a linear displacement of a particular point. FIG. 15C illustrates an example method for determining a severity of displacement based on the displacement of a point 1522 from its original (pre-distortion) position 1524. For example, the severity of displacement at point 1522 may be determined based on the magnitude of the displacement distance 1526 from original position 1524 to the current position of point 1522. For example, a quantitative measure Q may be determined according to the formula:

Q=ƒ(|gₐ(x)−g_{b}(x)|)

where gₐ(x) is the position of a point x after applying the distortion map, g_{b}(x) is the position of the same point x in the original image (i.e. before applying the distortion map), and ƒ is a function which takes the displacement of a point as an input. ƒ may be the identity function.

As yet another example, system 811 may determine the severity of the displacement at a particular point based on a local change in displacement. FIG. 15D illustrates an example method for determining a severity of displacement based on the local change in displacement of point 1522. The change in displacement at a particular point (e.g. point 1522) in a distorted image relative to nearby points can be a reasonable predictor of the severity of a subject's perceived distortion. That is, even if a point is severely displaced, if all surrounding points are similarly displaced then the subject may perceived relatively less distortion at that point. System 811 may determine a gradient (and/or an approximation thereof) over the distortion region, for example using vector analysis and/or numerical analysis methods. The gradient at a particular point is the rate of change in the point's displacement (as measured above) relative to nearby points, and/or instantaneously at that point. A quantitative measure Q for a particular point 1522 may be determined based on the gradient.

It may be convenient or otherwise desirable to approximate the local change in distortion for a particular point without necessarily conducting vector analysis on each point. The rate of change in displacement at a particular point 1522 (i.e. the approximated value of the gradient at point 1522) may be approximated based on the displacement distance 1526 (discussed above) and the inner distance 1536 between point 1522 and the boundary of the distortion region (e.g. at point 1532). Inner distance 1536 may be the distance from original position 1524 to boundary point 1532, the distance from the projection of point 1522 onto line 1514 to boundary point 1532, and/or some other distance which relates point 1522 to the boundary of the distortion region. Boundary point 1532 is the closest point to point 1522 on both the boundary of the distortion region and on line 1514 (or, equivalently in this example, line 1512). A quantitative measure may be determined by scaling the displacement distance 1526 by inner distance 1536 so that, if the displacement distances of points along line 1512 stay the same as their inner distances increase, the quantitative measure will reflect decreasing severity rather than uniform severity.

For example, a quantitative measure Q may be determined according to the formula:

$$Q = f ( \frac{{g}_{a} ( x ) - {g}_{b} ( x )}{d ( x )} )$$

where gₐ(x) is the position of point x after applying the distortion map, g_{b}(x) is the position of point x in the original image (i.e. before applying the distortion map), d(x) is the distance from the point x to the closest point on the boundary of the distortion region (e.g. inner distance 1536), and ƒ is a function which takes the displacement of a point as an input. ƒ may be the identity function.

In some embodiments, a quantitative measure Q may be based on the severity of the displacement of one or more points. For example, given a set of points P in the distortion region, the quantitative measure Q may be determined based on a statistical measure of the linear displacement and/or gradient of the points in P, such as (for example) the mean, median, mode, maximum, or other measure. Multiple quantitative measures Q may be determined by system 811; for instance, each point in P may be associated with its own quantitative measure, from which a “heat map” may be generated to assist in assessment of the severity of distortion in specific regions of the subject's vision.

### Interpretation of Terms

Unless the context clearly requires otherwise, throughout the description and the
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms.

Words that indicate directions such as “vertical”, “transverse”, “horizontal”, “upward”, “downward”, “forward”, “backward”, “inward”, “outward”, “vertical”, “transverse”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

Computer system 811 and/or components thereof (including, for example, one or more processors) may comprise hardware, software, firmware or any combination thereof. Computer system 811 may comprise one or more microprocessors, digital signal processors, graphics processors, field programmable gate arrays, and/or the like. Components of computer system 811 may be combined or subdivided, and components of computer system 811 may comprise sub-components shared with other components of computer system 811. Components of computer system 811 may be physically remote from one another.

Where a component is referred to above (e.g., a system, display, processor, etc.), unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

What is claimed is:

1. A method for mapping visual distortions perceived by a subject, the method performed by a processor in communication with a display, the method comprising:
displaying, at the display, a fixation target to the subject;
receiving, at the processor, an indication corresponding to an identification of a distortion region by the subject, the distortion region having a location determined relative to the fixation target;
displaying, at the display, an adjustable reference pattern to the subject, the adjustable reference pattern at least partially within the distortion region, the adjustable reference pattern adjustable within the distortion region and fixed outside the distortion region;
receiving, at the processor, an indication corresponding to an adjustment by the subject of the adjustable reference pattern within the distortion region, the adjustment at least partially complementary to visual distortion perceived by the subject in the distortion region;
determining, at the processor, a fixed reference pattern based on the adjustment of the adjustable reference pattern;
displaying, at the display, one or more adjustable patterns to the subject, each of the one or more adjustable patterns at least partially within the distortion region, each of the one or more adjustable patterns adjustable within the distortion region and fixed outside the distortion region;
receiving, at the processor, one or more indications corresponding to one or more adjustments by the subject of the one or more adjustable patterns within the distortion region, the one or more adjustments at least partially complementary to visual distortion perceived by the subject in the distortion region; and
determining, at the processor, a distortion map based on the one or more adjustments of the one or more adjustable patterns.

2. A method according to claim 1 wherein receiving an indication corresponding to an identification of a distortion region by the subject comprises receiving, at the processor, an indication corresponding to an identification by the subject of one or more boundaries, the distortion region bounded by the one or more boundaries.

3. A method according to claim 2 wherein the one or more boundaries comprise a plurality of straight lines.

4. A method according to claim 1 wherein displaying at least one of the adjustable reference pattern and the one or more adjustable patterns to the subject comprises displaying a plurality of graphical indicia, each of the graphical indicia within the distortion region being adjustable by the user.

5. A method according to claim 4 wherein the plurality of graphical indicia have a size which is selectable based on an indication by the subject.

6. A method according claim 4 wherein the plurality of graphical indicia are connected by a connecting line.

7. A method according to claim 4 wherein:
a first pattern of the one or more adjustable patterns comprises a first set of graphical indicia;
a second pattern of the one or more adjustable patterns comprises a second set of graphical indicia;
the graphical indicia of the first set comprise a first size and the graphical indicia of the second set comprising a second size larger than the first size; and
a distance between the first pattern and the fixation target is less than a distance between the second pattern and the fixation target.

8. A method according to claim 7 wherein:
a first region is associated with the first size and the second region is associated with the second size;
the first pattern at least partially overlaps a first region;
the second pattern at least partially overlaps a second region and is entirely outside the first region;
a size of the graphical indicia of the first set is selected based on a determination that the first pattern overlaps the first region; and
a size of the graphical indicia of the first set is selected based on a determination that the second pattern overlaps the second region.

9. A method according to claim 8 wherein the first set of graphical indicia comprise at least the graphical indicia of the first pattern which overlap with the first region and the second set of graphical indicia comprise at least the graphical indicia of the second pattern which overlap with the second region.

10. A method according to claim 1 comprising:
displaying, at the display, a validation pattern based on the distortion map;
temporally varying the validation pattern at least in the distortion region; and
in response to receiving an indication that the adjusted pattern does not meet a validation criterion, receiving a further adjustment of at least one of the one or more adjustable patterns.

11. A method according to claim 10 wherein temporally varying comprises rotating the validation pattern and, at each step of the rotation of the validation pattern, displaying the validation pattern based on the distortion map so that the validation pattern is only distorted in the distortion region.

12. A method according to claim 1 comprising determining a perception map based on the distortion map, the perception map approximating the distortion perceived by the subject, the perception map being an inverse of the distortion map.

13. A method according to claim 12 wherein the perception map maps positions in a first space to position in a second space, the distortion map maps elements from positions in the second space to positions in the first space, and, for a first position in the first space and a second position in the second space, the perception map is determined to map the first position to the second position based on the distortion map mapping the second position to the first position.

14. A method according to claim 1 comprising determining, at the processor, a quantitative measure based on the one or more adjustments of the one or more adjustable patterns for quantifying the severity of distortions perceived by the subject.

15. A method according to claim 14 wherein determining a quantitative measure comprises:
identifying, at the processor, an initial pattern at least partially overlapping with the distortion region;
identifying, at the processor, a distorted pattern based on the initial pattern and at least one of the one or more adjustments;
determining, at the processor, a displacement area between the initial pattern and the distorted pattern;
determining, at the processor, the quantitative measure based on the displacement area.

16. A method according to claim 1 wherein determining a quantitative measure comprises:
identifying, at the processor, an initial point in the distortion region;
identifying, at the processor, a distorted point based on the initial point and at least one of the one or more adjustments;
determining, at the processor, a severity of displacement between the initial point and the distorted point;
determining, at the processor, the quantitative measure based on the severity of displacement.

17. A method according to claim 16 wherein determining a severity of displacement comprises:
determining, at the processor, a gradient corresponding to a rate of change between a plurality of adjustments of the one or more adjustments; and
determining, at the processor, the quantitative measure based on a value of the gradient at the initial point.

18. A method according to claim 1 comprising:
determining, at the processor, a viewing distance between the subject and a surface of the display;
determining, at the processor, a display size of the display;
displaying, at the processor, at least one of the adjustable reference pattern, the fixed reference pattern, and the one or more adjustable patterns to the subject based on the viewing distance and the display size;
wherein:
at least one of the adjustable reference pattern, the fixed reference pattern, and the one or more adjustable patterns are associated with an angular size; and
displaying at least one of the adjustable reference pattern, the fixed reference pattern, and the one or more adjustable patterns to the subject based on the viewing distance and the display size comprises:
determining a linear size based on the angular size, viewing distance, and display size so that the linear size occupies a portion of the subject's field of view corresponding to the angular size; and
displaying, at the processor, at least one of the adjustable reference pattern, the fixed reference pattern, and the one or more adjustable patterns to the subject based on the linear size.

19. A method according to claim 1 comprising:
selecting an intersection point from among a plurality of points, each of the plurality of points being in the distortion region, the intersection point being no further from the fixation target than any other point in the plurality of points;
wherein displaying an adjustable reference pattern to the subject comprises displaying first and second adjustable reference pattern portions, the second adjustable reference pattern portion intersecting with the first adjustable reference pattern portion at the intersection point.

20. A system for mapping visual distortions perceived by a subject, the system comprising:
a display;
a processor in communication with the display, the processor configured to:
drive the display to display a fixation target to the subject;
receive an indication corresponding to an identification of a distortion region by the subject, the distortion region having a location determined relative to the fixation target;
drive the display to display an adjustable reference pattern to the subject, the adjustable reference pattern at least partially within the distortion region, the adjustable reference pattern adjustable within the distortion region and fixed outside the distortion region;
receive an indication corresponding to an adjustment by the subject of the adjustable reference pattern within the distortion region, the adjustment at least partially complementary to visual distortion perceived by the subject in the distortion region;
determine a fixed reference pattern based on the adjustment of the adjustable reference pattern;
drive the display to display one or more adjustable patterns to the subject, each of the one or more adjustable patterns at least partially within the distortion region, each of the one or more adjustable patterns adjustable within the distortion region and fixed outside the distortion region;
receive one or more indications corresponding to one or more adjustments by the subject of the one or more adjustable patterns within the distortion region, the one or more adjustments at least partially complementary to visual distortion perceived by the subject in the distortion region; and
determine a distortion map based on the one or more adjustments of the one or more adjustable patterns.
